(19) United States
(12) Patent Application Publication
(10) Pub. No.: US 2026/0165837 A1
(43) Pub. Date: Jun. 18, 2026

(54) PRE-DILATED EXPANDABLE SHEATH WITH PROTECTIVE COVER

(71) Applicant: Edwards Lifesciences Corporation, Irvine, CA (US)

(72) Inventors: Izaak Rosen, Irvine, CA (US); Sean Chow, Irvine, CA (US); Timothy C. Ulrich, Irvine, CA (US); Jeffrey S. Kasalko, Irvine, CA (US)

(73) Assignee: Edwards Lifesciences Corporation, Irvine, CA (US)

(21) Appl. No.: 19/415,592

(22) Filed: Dec. 10, 2025

Related U.S. Application Data
(63) Continuation of application No. PCT/US2024/034914, filed on Jun. 21, 2024.
(60) Provisional application No. 63/522,472, filed on Jun. 22, 2023.

(51) Int. Cl. A61F 2/24; A61M 25/00

(52) U.S. Cl. CPC A61F 2/2436; A61F 2/243; A61M 25/0009; A61M 2209/06

(57) ABSTRACT

Various implementations include an expandable sheath system including a radially expandable sheath including a restraining member limiting expansion of the sheath and methods of making the same.

### CROSS-REFERENCE TO RELATED APPLICATIONS

This application is a continuation of International Application No. PCT/US2024/034914, filed Jun. 21, 2024, which claims the benefit of U.S. Provisional Application No. 63/522,472, filed Jun. 22, 2023, the contents of which are incorporated herein by reference in their entirety.

### FIELD

The present application is directed to a sheath for use with catheter-based technologies for repairing and/or replacing heart valves, as well as for delivering an implant, such as a prosthetic valve to a heart via the patient's vasculature.

### BACKGROUND

Endovascular delivery catheter assemblies are used to implant prosthetic devices, such as a prosthetic valve, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques.

Percutaneous interventional medical procedures utilize the large blood vessels of the body to reach target destinations rather than surgically opening the target site. There are many types of disease states that can be treated via interventional methods including coronary blockages, valve replacements (TAVR) and brain aneurysms. These techniques involve using wires, catheters, balloons, electrodes and other thin devices to travel down the length of the blood vessels from the access site to the target site. The devices have a proximal end which the clinician controls outside of the body and a distal end inside the body which is responsible for treating the disease state. Percutaneous interventional procedures offer several advantages over open surgical techniques. First, they require smaller incision sites which reduces scarring and bleeding as well as infection risk. Percutaneous procedures are also less traumatic to the tissue, so recovery times are reduced. Finally, percutaneous interventional techniques can usually be performed much faster, and with fewer clinicians participating in the procedure, so overall costs are lowered. In some cases, the need for anesthesia is also eliminated, further speeding up the recovery process and reducing risk.

A single procedure typically uses several different guidewires, catheters, and balloons to achieve the desired effect. One at a time, each tool is inserted and then removed from the access site sequentially. For example, a guidewire is used to track to the correct location within the body. Next a balloon may be used to dilate a section of narrowed blood vessel. Last, an implant may be delivered to the target site. Because catheters are frequently inserted and removed, introducer sheaths are used to protect the local anatomy and simplify the procedure.

An introducer sheath can be used to safely introduce a delivery apparatus into a patient's vasculature (for example, the femoral artery). Introducer sheaths are conduits that seal onto the access site blood vessel to reduce bleeding and trauma to the vessel caused by catheters with rough edges. An introducer sheath generally has an elongated sleeve that is inserted into the vasculature and a housing that contains one or more sealing valves that allow a delivery apparatus to be placed in fluid communication with the vasculature with minimal blood loss. Once the introducer sheath is positioned within the vasculature, the shaft of the delivery apparatus is advanced through the sheath and into the vasculature, carrying the prosthetic device. Expandable introducer sheaths, formed of highly elastomeric materials, allow for the dilating of the vessel to be performed by the passing prosthetic device.

The force required to advance a delivery system/medical device through a sheath is attributed to the frictional forces between the sheath and the delivery device, the force required to radially expand the sheath, and the force required to expand the adjacent patient anatomy, for example, the femoral vessel. High push forces result in physician fatigue and errors and increase the time needed to complete a procedure.

The push force required to advance a delivery system and/or medical device through the sheath depends on a variety of factors, including patient anatomy, crimped valve profile/delivery system profile, and the sheath design/manufacturing. For example, the expandable sheath, formed of highly elastomeric materials and some including one or more folds to aid in expansion, expands as an implantable device is inserted through the sheath. These sheaths sometimes include a strain relief portion that extends along/over the outer surface of the sheath (for example, at the proximal end) and forms a smooth transition from the sheath hub to the sheath. The strain relief portion restricts expansion of the underlying sheath and helps to ensure hemostasis between the portions of the sheath inside the patient and the sheath hub (external to the patient). Because the strain relief portion resists expansion, higher push forces are required as the delivery device/system and implant are introduced into and advanced through the sheath/strain relief portion. In addition, recent trends in heart valves including thicker PVL skirts have increased the crimped profile of the heart valve/delivery device and can lead to even higher push forces through the sheath, and particularly the strain relief portion.

One method to reduce push forces required to advance the delivery device through the sheath is to pre-dilate the sheath and/or strain relief portion by passing a relatively large dilator (for example, 22 French dilator) into the sheath. This is done during sheath prep, prior to sheath insertion into the patient and/or with the sheath at least partially inserted into the patient. The challenge with this method is that it can be difficult with regard to physical strength of the user (i.e., grip and arm strength) to advance the dilator into sheath. Additionally, it is important that the dilator pass all the way to the distal end of the sheath while also avoiding splitting the sheath and/or distal end of the sheath, which could cause difficulty or vessel injury during the delivery device insertion/removal process.

Accordingly, there remains a need for devices, systems, and methods of providing a sheath including a strain relief portion, that allows the sheath body to expand, reducing the initial push force when introducing the delivery system and implant.

### SUMMARY

Implementations of the present expandable sheath system can minimize trauma to the vessel and damage to the sheath and prosthetic device by reducing push forces through the sheath. Some implementations ensure that the sheath is not damaged in an effort to dilate or expand the strain relief portion. Some implementations can comprise a sheath with a smaller profile than that of prior art introducer sheaths. Furthermore, some implementations can reduce the length of time a procedure takes, as well as reduce the risk of a longitudinal or radial vessel tear, or plaque dislodgement because less push force is required and only one sheath is used, rather than several different sizes of sheaths.

An implementation of the present disclosure method of manufacturing a pre-dilated expandable sheath for delivering a medical device. In one of its basic configurations/implementations, the present disclosure provides a method including: providing a radially expandable sheath having a tubular strain relief layer; providing a restraining member over the sheath for limiting expansion; dilating the sheath; and removing the restraining member. This basic configuration/implementation can preferably be provided with any one or more of the features described elsewhere herein, in particular with those of the examples described hereafter. However, it should be understood that the basic configuration/implementation can preferably also be provided with any one or more of the features shown in the figures and/or described in conjunction with the figures, either in addition to or alternatively to the features of the examples described hereafter.

In some implementations, the method includes providing a radially expandable sheath including: a continuous inner layer defining a lumen therethrough, the inner layer including a proximal portion and a main body portion, and having a folded portion extending along a length of the inner layer; a tubular strain relief layer provided over the proximal portion of the inner layer; wherein at least a portion of the sheath is configured to locally expand from an unexpanded configuration in which the lumen has a first diameter to an expanded configuration in which the lumen has a second, larger, diameter (for example, in response to an outwardly directed radial force exerted on the lumen by an expansion element of a dilator and/or medical device), and then locally contract at least partially back toward unexpanded configuration (for example, as the dilator and/or medical device passes through the lumen).

In some implementations, the method includes providing a restraining member positioned over a distal end of the strain relief layer, the restraining member limiting expansion of an adjacent (for example, underlying) portion at least one of the inner layer and strain relief layer. For example, in some implementations, the restraining member limits expansion/unfolding of the folded portion thereby preventing the seam from propagating axially or from further opening radially after pre-expansion.

In some implementations, the method includes introducing a dilator into a proximal end of the lumen of the sheath, the dilator including an expansion element provided thereon.

In some implementations, the method includes advancing the dilator through the proximal portion of the inner layer such that the expansion element provided on the dilator exerts an outwardly directed radial force against the lumen and causes the inner layer (and/or the strain relief layer) proximate the expansion element to locally expand from an unexpanded configuration to an expanded configuration.

In some implementations, the restraining member limits expansion of the sheath (for example, inner layer and strain relief layer) proximate the restraining member.

In some implementations, the method includes heating the sheath. For example, in some implementations, the method includes sterilizing the sheath and/or heat setting the sheath in the expanded-folded configuration.

In some implementations, the restraining member limits the unfolding of the folded portion of the inner layer proximate the restraining member when the sheath moves from the unexpanded configuration to the expanded configuration during advancement of the dilator through the proximal portion of the inner layer.

In some implementations, the restraining member is provided over a length of sheath at a location corresponding to the distal end of the strain relief layer and extends along a length of the strain relief layer from the distal end toward a proximal end of the strain relief layer, and along a second length of the sheath from the distal end of the strain relief layer toward a distal end of the sheath.

In some implementations, providing the restraining member over the sheath includes coupling (for example, releasably coupling) the restraining member to at least one of the inner layer or the strain relief layer.

In some implementations, an inner surface of the restraining member includes an adhesive (for example, temporary/releasable adhesive) for coupling the restraining member to the sheath.

In some implementations, the restraining member includes a shrink tubing, where coupling the restraining member to at least one of the inner layer or the strain relief layer includes providing a shrink process (for example, a shrink heating process) to the restraining member.

In some implementations, the method further includes removing the restraining member from the inner layer and the strain relief layer.

In some implementations, the restraining member is removed after the heating step. For example, in some implementations, the restraining member is removed immediately before the medical procedure.

In some implementations, the restraining member is removed before the heating step.

In some implementations, a release feature is incorporated into a packaging sized and configured to receive the sheath, where providing a radially expandable sheath includes removing the sheath from the packaging, wherein removing the sheath from the packaging removes the restraining member from the inner layer and the strain relief layer.

In some implementations, advancing the dilator through the proximal portion of the inner layer includes advancing the expansion element of the dilator aligns with the distal end of the strain relief layer, such that the distal end of the strain relief layer is expanded. For example, in some implementations, the proximal end of the tapered portion and/or a distal end of the body portion of a dilator shaft aligns with the distal end of the strain relief layer.

In some implementations, advancing the dilator through the proximal portion of the inner layer includes advancing the expansion element of the dilator to beyond the distal end of the strain relief layer, such that the distal end of the strain relief layer and a portion of the main body portion of the inner layer is expanded. For example, in some implementations, the expansion element is used to expand/dilate a length of the main body portion of the sheath extending 10-15 mm beyond the distal end of the strain relief layer.

In some implementations, expanding the portion of the main body portion beyond the strain relief layer causes a corresponding length of the folded portion to at least partially unfold. For example, in some implementations, expanding the portion of the main body portion beyond the strain relief layer causes any bonding between the folded layers of the inner layer of the sheath to separate.

In some implementations, the method further includes removing the dilator from the lumen of the sheath after the heating step is complete. In some implementations, the dilator remains within the sheath during the heating step.

In some implementations, the method further includes removing the dilator from the lumen of the sheath before the heating step.

In some implementations, at least a portion of the strain relief layer is configured to locally expand from an unexpanded configuration at a first diameter to an expanded configuration at a second, larger, diameter (for example, due to an outwardly directed radial force exerted on the lumen of the inner layer by the dilator and/or a medical device against the inner layer), and then locally contract at least partially back to the unexpanded configuration (for example, as the dilator and/or medical device passes through the lumen).

In some implementations, at least a portion of the strain relief layer is configured to locally expand from the unexpanded configuration to the expanded configuration in response to the outwardly directed radial force exerted against the lumen (for example, inner layer) by the dilator, and then locally contract at least partially back to the unexpanded configuration as the dilator moves within the lumen, wherein at least a portion of the sheath is configured to locally expand from the unexpanded configuration to the expanded configuration in response to an outwardly directed radial force exerted on the lumen of the inner layer by the dilator, and then locally contract at least partially back to the unexpanded configuration as the dilator moves within the lumen.

In some implementations, the sheath further includes: an outer layer provided over the inner layer, where the outer layer is discontinuous and includes an overlapping portion and an underlying portion, and the overlapping portion overlaps the underlying portion, wherein at least a portion of the folded portion of the inner layer is positioned between the overlapping portion and the underlying portion, wherein the strain relief layer extends at least partially over the outer layer.

In some implementations, in the unexpanded configuration, the folded portion extends circumferentially over an outer surface of the inner layer and/or outer layer, wherein, in the expanded configuration, local expansion causes a length of the folded portion to at least partially unfold, wherein, in the expanded configuration, local expansion of the sheath causes a length of the overlapping portion to move circumferentially with respect to the underlying portion.

In some implementations, in the expanded configuration, local expansion of the sheath forms a gap between longitudinally extending edges of the outer layer, wherein at least a portion of the unfolded portion extends into the gap, wherein the restraining member limits expansion of the sheath and a width of the gap proximate the restraining member.

In some implementations, the sheath further includes an elastic outer cover extending at least partially over the sheath (for example, at least partially over the inner layer, the outer layer, and/or the strain relief layer, and under the restraining member when included) where the outer cover locally expands and contracts as the medical device is advanced through the lumen, wherein the elastic outer cover exerts a radially inward force on the sheath (for example, urging the inner layer, outer layer, and or strain relief layer toward the unexpanded configuration).

In some implementations, the sheath further includes a sheath hub fixedly coupled to the proximal end of the sheath, the sheath hub including a central lumen extending therethrough and coaxial with the lumen of the sheath, wherein the dilator shaft is sized and configured to be received (for example, slidably received) within the central lumen of the sheath hub, wherein the dilator includes a dilator hub coupled to a proximal end of the dilator shaft, wherein the method further includes: advancing the dilator through the proximal portion until the dilator hub abuts the sheath hub; coupling the dilator hub to the sheath hub before heating the sheath. For example, in some implementations, the dilator hub is coupled to the sheath hub by a press fit, an interference fit, a snap fit, a pin, thread, bayonet fastener, clip, and/or locking key.

In some implementations, heating the sheath includes heating the sheath at a temperature and for a duration corresponding to a sterilization process, wherein, during the heating, the sheath is not heated at a temperature or for a duration sufficient to bond layers of the folded portion.

In some implementations, heating the sheath includes heating the sheath at a temperature of 60° C.

In some implementations, heating the sheath includes heating the sheath for a duration greater than 12 hours (for example, at 60° C. for 24 hours, at 60° C. for 26 hours).

A further implementation of the present disclosure a sheath system including: a radially expandable sheath including: a continuous inner layer defining a lumen therethrough, the inner layer including a proximal portion and a main body portion and a folded portion extending along a length of the inner layer. In some implementations, the expandable sheath includes a tubular strain relief layer provided over the proximal portion of the inner layer. In some implementations, the expandable sheath includes a restraining member positioned over a distal end of the strain relief layer, the restraining member limiting expansion of an adjacent (for example, underlying) portion of at least one of the inner layer and strain relief layer (for example, limiting expansion/unfolding of the folded portion thereby preventing the seam formed between the outer layer and the inner layer and/or between adjacent layers of the folded portion from propagating axially or from further opening radially after pre-expansion). In some implementations, the sheath system further includes a dilator sized and configured to be received within the lumen of the inner layer, the dilator including an elongated dilator shaft and an expansion element provided thereon. In some implementations, at least a portion of the sheath (for example, inner layer and/or strain relief layer) is configured to locally expand from an unexpanded configuration in which the lumen has a first diameter to an expanded configuration in which the lumen has a second, larger, diameter in response to an outwardly directed radial force exerted on the lumen by the expansion element of the dilator (and/or medical device), and then locally contract at least partially back toward unexpanded configuration as the dilator (and/or medical device) passes through the lumen. In some implementations, the restraining member limits expansion of the sheath (for example, inner layer and/or strain relief layer) proximate the restraining member.

In some implementations, the restraining member limits the unfolding of the folded portion of the inner layer proximate the restraining member when the sheath moves from the unexpanded to the expanded configuration.

In some implementations, the restraining member is provided over a length of sheath at a location corresponding to the distal end of the strain relief layer and extends along a length of the strain relief layer from the distal end toward a proximal end of the strain relief layer, and along a second length of the sheath from the distal end of the strain relief layer toward a distal end of the sheath.

In some implementations, the restraining member comprises at least one of a tape, a shrink tube, an elastic tube, or a packaging feature.

In some implementations, the restraining member is coupled to (for example, releasably coupling) the sheath. For example, in some implementations, the restraining member is coupled to the inner layer and/or strain relief layer.

In some implementations, an inner surface of the restraining member includes an adhesive (for example, temporary/releasable adhesive) for coupling the restraining member to the sheath.

In some implementations, the restraining member includes a shrink tubing, where the restraining member is coupled to at least one of the inner layer or the strain relief layer by a shrink process (for example, a shrink heating process).

In some implementations, the restraining member includes a release feature for removing the restraining member from the sheath (for example, inner layer and/or the strain relief layer).

In some implementations, the release feature includes at least one of a weakened portion or a pull tab and/or line integral with the restraining member. For example, in some implementations, the release feature includes a perforation, scoreline, and/or slit.

In some implementations, the release feature is incorporated into a packaging sized and configured to receive the sheath, where removing the sheath from the packaging removes the restraining member from the inner layer and the strain relief layer.

In some implementations, the dilator shaft includes a body portion adjacent a proximal end of the dilator shaft, and a tapered portion extending from a distal end of the dilator shaft toward the body portion, where the expansion element is provided on the body portion.

In some implementations, the expansion element is defined by the body portion of the dilator shaft.

In some implementations, the expansion element includes a projection extending from an outer surface of the dilator shaft. For example, in some implementations, the expansion element can include a regular or irregular shaped projection extending from the outer surface of the dilator shaft. For example, in some implementations, the projection extends around all or a portion of the circumference of the dilator shaft.

In some implementations, the diameter of the expansion element is 22F. For example, in some implementations, the expansion element of dilator has a diameter ranging from 12F to 24F, from 14F to 24F, from 14F to 22F.

In some implementations, at least a portion of the strain relief layer is configured to locally expand from an unexpanded configuration at a first diameter to an expanded configuration at a second, larger, diameter (for example, due to an outwardly directed radial force exerted on the lumen of the inner layer by the dilator and/or a medical device against the inner layer), and then locally contract at least partially back to the unexpanded configuration (for example, as the dilator and/or medical device passes through the lumen).

In some implementations, at least a portion of the strain relief layer is configured to locally expand from the unexpanded configuration to the expanded configuration in response to an outwardly directed radial force exerted against the lumen (for example, inner layer) by the dilator, and then locally contract at least partially back to the unexpanded configuration as the dilator moves within the lumen.

In some implementations, the strain relief layer includes: a proximal portion adjacent a proximal end of the strain relief layer; a distal portion adjacent a distal end of the strain relief layer; and a tapered portion extending between the distal portion and the proximal portion, wherein a diameter of the proximal portion is greater than a diameter of the distal portion.

In some implementations, the strain relief layer comprises a stiffer and/or less elastomeric material than the inner layer and restricts expansion of the inner layer.

In some implementations, the strain relief layer comprises a material having a higher durometer than the inner layer such that the strain relief layer restricts expansion of the sheath (for example, inner and/or outer layer).

In some implementations, the strain relief layer comprises polyurethane. For example, in some implementations, the strain relief layer comprises high density polyethylene.

In some implementations, as the strain relief layer moves from the unexpanded configuration to the expanded configuration, a length of the strain relief layer remains constant.

In some implementations, the sheath further includes: an outer layer provided over the inner layer; wherein the strain relief layer comprises a stiffer and/or less elastomeric material than the inner layer and outer layer and restricts expansion of at least one of the inner or outer layers, wherein the strain relief layer comprises a material having a higher durometer than the inner layer and/or the outer layer such that the strain relief layer restricts expansion of at least one of the inner or outer layers.

In some implementations, the sheath further includes: an outer layer provided over the inner layer, where the outer layer is discontinuous and includes an overlapping portion and an underlying portion, and the overlapping portion overlaps the underlying portion, where at least a portion of the folded portion of the inner layer is positioned between the overlapping portion and the underlying portion, where the strain relief layer extends at least partially over the outer layer.

In some implementations, in the unexpanded configuration, the folded portion extends circumferentially over an outer surface of the inner layer and/or outer layer.

In some implementations, in the expanded configuration, local expansion causes a length of the folded portion to at least partially unfold forming an unfolded portion of the inner layer, where in the expanded configuration, local expansion of the sheath causes a length of the overlapping portion to move circumferentially with respect to the underlying portion.

In some implementations, in the expanded configuration, local expansion of the sheath forms a gap between longitudinally extending edges of the outer layer, wherein at least a portion of the unfolded portion extends into the gap, where the restraining member limits expansion of the sheath and a width of the gap proximate the restraining member.

In some implementations, an overall length of the strain relief layer and/or sheath does not change when the sheath and/or strain relief layer moves between the unexpanded configuration and expanded configuration.

In some implementations, the lumen of the inner layer is cylindrical in the unexpanded and expanded configurations.

In some implementations, the inner layer comprises PTFE and the outer layer comprises HDPE and/or Tecoflex.

In some implementations, the inner and outer layers are bonded together.

In some implementations, the inner and outer layers are thermally bonded together.

In some implementations, the inner and outer layers are bonded together by an adhesive.

In some implementations, the strain relief layer is bonded to the outer layer and/or inner layer.

In some implementations, the strain relief layer is thermally and/or adhesively bonded to the outer layer and/or inner layer.

In some implementations, the inner layer comprises a woven fabric and/or braided filaments.

In some implementations, the inner layer comprises yarn filaments of PTFE, PET, PEEK, and/or nylon.

In some implementations, the outer layer comprises polyurethane (for example, high density polyethylene).

In some implementations, the sheath system further includes an elastic outer cover extending at least partially over the sheath (for example, at least partially over the inner layer, the outer layer, and/or the strain relief layer), where the outer cover locally expands and contracts as the dilator (and/or medical device) is advanced through the lumen. In some implementations, the elastic outer cover exerts a radially inward force on the sheath (for example, urging the inner layer, outer layer, and or strain relief layer toward the unexpanded configuration). In some implementations, the elastic outer cover comprises PEBAX, polyurethane, silicone, or polyisoprene, or combination thereof.

In some implementations, the sheath further includes a sheath hub fixedly coupled to the proximal end of the sheath. In some implementations, the sheath hub includes a central lumen extending therethrough and coaxial with the lumen of the sheath, wherein the dilator shaft is sized and configured to be received (for example, slidably received) within the central lumen of the sheath hub. In some implementations, the dilator includes a dilator hub coupled to a proximal end of the dilator shaft, where the dilator hub is configured to be coupled to the sheath hub. For example, in some implementations, the dilator hub is coupled to the sheath hub by a press fit, an interference fit, a snap fit, a pin, thread, bayonet fastener, clip, and/or locking key.

In some implementations, the sheath hub includes one or more seals for forming a seal around an outer surface of a delivery apparatus movable through the central lumen of the sheath hub.

Another implementation of the present disclosure provides sheath kit system including: a radially expandable sheath including. In some implementations, the expandable sheath includes a continuous inner layer defining a lumen therethrough, the inner layer including a proximal portion and a main body portion and a folded portion extending along a length of the inner layer. In some implementations, the expandable sheath includes a tubular strain relief layer provided over the proximal portion of the inner layer and a restraining member positioned over a distal end of the strain relief layer, the restraining member limiting expansion of an adjacent (for example, underlying) portion of at least one of the inner layer and strain relief layer. For example, in some implementations, the restraining member limits expansion/unfolding of the folded portion thereby preventing the seam from propagating axially or from further opening radially after pre-expansion. In some implementations, the sheath kit system further includes a dilator sized and configured to be received within the lumen of the inner layer, the dilator including an elongated shaft and an expansion element provided thereon. In some implementations, the sheath kit system includes a tray sized and configured to receive the sheath and the dilator, the tray including a release mechanism coupled to the restraining member, wherein upon removal of the sheath from the tray, the release mechanism retains the restraining member thereby removing it from the sheath. In some implementations, at least a portion of the sheath (for example, inner layer and/or strain relief layer) is configured to locally expand from an unexpanded configuration in which the lumen has a first diameter to an expanded configuration in which the lumen has a second, larger, diameter in response to an outwardly directed radial force exerted on the lumen by the expansion element of the dilator (and/or medical device), and then locally contract at least partially back toward unexpanded configuration as the dilator (and/or medical device) passes through the lumen. In some implementations, the restraining member limits expansion of the sheath (for example, inner layer and strain relief layer) proximate the restraining member.

A further implementation of the present disclosure provides a method of delivering a medical device through a sheath. In some implementations, the method includes providing a radially expandable sheath having a continuous inner layer defining a lumen therethrough, where the inner layer includes a proximal portion and a main body portion and a folded portion extending along a length of the inner layer. In some implementations, the expandable sheath includes a tubular strain relief layer provided over the proximal portion of the inner layer and a restraining member positioned over a distal end of the strain relief layer, where the restraining member limiting expansion of an adjacent (for example, underlying) portion of at least one of the inner layer and strain relief layer. For example, in some implementations, the restraining member limits expansion/unfolding of the folded portion thereby preventing the seam from propagating axially or from further opening radially after pre-expansion. In some implementations, the method further includes removing a dilator received from the lumen of the inner layer, where the restraining member limits expansion of the sheath (for example, inner layer and/or strain relief layer) due to an outwardly directed radial force exerted by the dilator. In some implementations the method includes removing the restraining member from the sheath. In some implementations the method includes introducing a medical device into a proximal end of a central lumen of the sheath; advancing the medical device through the proximal portion of the inner layer (for example, the portion of the sheath corresponding to the strain relief layer) and thereby exerting an outwardly directed radial force by the medical device against the central lumen (for example, inner layer) causing the inner layer and the strain relief layer proximate the medical device to locally expand from an unexpanded configuration to an expanded configuration, and locally contracting the strain relief layer towards the unexpanded configuration as the medical device passes through the corresponding portion of the lumen of sheath. In some implementations, the method includes advancing the medical device beyond a distal end of the strain relief layer and advancing a medical device through the main body portion of the lumen of the sheath causing the main body portion of the sheath to locally expand from the unexpanded configuration to the expanded configuration at a location proximate the medical device in response to the outwardly directed radial force of the medical device exerted against the inner layer locally contracting the sheath at least partially back to the unexpanded configuration as the medical device passes through the lumen, and advancing the medical device beyond a distal opening in the sheath.

In some implementations, at least a portion of the sheath (for example, inner layer and/or strain relief layer) is configured to locally expand from an unexpanded configuration in which the lumen has a first diameter to an expanded configuration in which the lumen has a second, larger, diameter in response to an outwardly directed radial force exerted on the lumen by the expansion element of the dilator (and/or medical device), and then locally contract at least partially back toward unexpanded configuration as the dilator (and/or medical device) passes through the lumen.

Yet another implementation of the present disclosure provides a method of inserting a medical device into a blood vessel of a patient. In some implementations, the method includes providing a radially expandable sheath having a continuous inner layer defining a lumen therethrough, the inner layer including a proximal portion and a main body portion and a folded portion extending along a length of the inner layer. In some implementations, the expandable sheath includes a tubular strain relief layer provided over the proximal portion of the inner layer and a restraining member positioned over a distal end of the strain relief layer, where the restraining member limiting expansion of an adjacent (for example, underlying) portion of at least one of the inner layer and strain relief layer. For example, in some implementations, the restraining member limits expansion/unfolding of the folded portion thereby preventing the seam from propagating axially or from further opening radially after pre-expansion. In some implementations, the method further includes removing a dilator received from the lumen of the inner layer, where the restraining member limits expansion of the sheath (for example, inner layer and/or strain relief layer) due to an outwardly directed radial force exerted by the dilator. In some implementations the method includes removing the restraining member from the sheath. In some implementations the method includes inserting the sheath at least partially into the blood vessel of the patient; introducing a medical device into a proximal end of the central lumen of the sheath. In some implementations the method includes advancing the medical device through the proximal portion of the inner layer (for example, the portion of the sheath corresponding to the strain relief layer) and thereby exerting an outwardly directed radial force by the medical device against the central lumen (for example, inner layer) causing the inner layer and the strain relief layer proximate the medical device to locally expand from an unexpanded configuration to an expanded configuration, and locally contracting the strain relief layer towards the unexpanded configuration as the medical device passes through the corresponding portion of the lumen of sheath; advancing the medical device beyond the distal end of the strain relief layer. In some implementations the method includes advancing a medical device through the main body portion lumen of the sheath causing the main body portion of the sheath to locally expand from an unexpanded configuration to an expanded configuration at a location proximate the medical device in response to the outwardly directed radial force of the medical device exerted against the inner layer, and locally contracting the sheath at least partially back to the unexpanded configuration as the medical device passes through the lumen; and advancing the medical device beyond a distal opening in the sheath to a treatment site within the blood vessel.

In some implementations, the dilator expands the distal end of the strain relief layer.

In some implementations, the inner layer includes at least one folded portion, wherein locally expanding the lumen of the sheath causes a length of the folded portion to at least partially unfold.

In some implementations, the sheath further includes: an outer layer provided over the inner layer, where the outer layer is discontinuous and includes an overlapping portion and an underlying portion.

In some implementations, when the sheath is in the unexpanded configuration, the overlapping portion overlaps the underlying portion with the folded portion of the inner layer disposed between the overlapping portion and the underlying portion,

In some implementations, the strain relief layer extends at least partially over the outer layer.

In some implementations, the medical device is a prosthetic device mounted in a radially crimped state on a delivery apparatus.

In some implementations, advancing the prosthetic device through the lumen of the sheath comprises advancing the delivery apparatus and the prosthetic device through the lumen of the sheath and into a vasculature of the patient.

In some implementations, the prosthetic device comprises a prosthetic heart valve and the method further comprises implanting the prosthetic heart valve at the treatment site within the patient.

In some implementations, the prosthetic heart valve is mounted on a balloon catheter of the delivery apparatus as the prosthetic heart valve is advanced through the sheath.

In some implementations, the sheath is inserted into a femoral artery of the patient.

Various aspects of the implementations described above can be combined based on desired sheath system characteristics.

### DESCRIPTION OF DRAWINGS

FIG. 1 is an elevation view of an expandable sheath along with an endovascular delivery apparatus for implanting a prosthetic implant.

FIG. 2 is an elevation view of an expandable sheath including an introducer locking hub, a sheath locking sleeve, and an introducer.

FIG. 3 is an elevation view of the expandable sheath of FIG. 2 along with an endovascular delivery apparatus for implanting a prosthetic implant.

FIG. 4 is an elevation view of an expandable sheath a sheath hub, an introducer locking hub, and a sheath locking sleeve of FIG. 2.

FIG. 5A is a cross sectional view of the sheath hub, introducer locking hub, and sheath locking sleeve of FIG. 2.

FIG. 5B is a cross sectional view of the introducer cap, the sheath hub, the introducer locking hub, the sheath locking sleeve of FIG. 2.

FIG. 6 is a cross sectional view of the introducer cap, sheath hub, introducer locking hub, and sheath locking sleeve of FIG. 2.

FIG. 7 is a distal end view of the sheath locking sleeve of FIG. 2 and the proximal fluid seal of FIGS. 5A-B.

FIG. 8A is a first elevation view of the introducer locking hub of FIG. 2 coupled to an introducer.

FIG. 8B is a second (rotated) elevation view of the introducer locking hub of FIG. 2 coupled to the introducer.

FIG. 8C is a distal end view of the introducer locking hub of FIG. 2 coupled to the introducer.

FIG. 8D is a partial side view of the introducer locking hub of FIG. 2 coupled to the introducer.

FIG. 8E is a partial perspective view of the introducer locking hub of FIG. 2 coupled to the introducer.

FIG. 8F is a partial perspective view of the introducer locking hub of FIG. 2 coupled to the introducer.

FIG. 9A is a distal end view of the introducer locking hub of FIG. 2.

FIG. 9B is a first elevation view of the introducer locking hub of FIG. 2.

FIG. 9C is a proximal end view of the introducer locking hub of FIG. 2.

FIG. 9D is a first perspective view of the introducer locking hub of FIG. 2.

FIG. 9E is a second elevation view of the introducer locking hub of FIG. 2.

FIG. 9F is a second perspective view of the introducer locking hub of FIG. 2.

FIG. 10A is a distal end view of the sheath locking sleeve of FIG. 2.

FIG. 10B is a first elevation view of the sheath locking sleeve of FIG. 2.

FIG. 10C is a proximal end view of the sheath locking sleeve of FIG. 2.

FIG. 10D is a first perspective view of the sheath locking sleeve of FIG. 2.

FIG. 10E is a second elevation view of the sheath locking sleeve of FIG. 2.

FIG. 10F is a second perspective view of the sheath locking sleeve of FIG. 2.

FIG. 11 is a side elevation cross-sectional view of a portion of the expandable sheath of FIGS. 1 and 2.

FIG. 12 is a magnified view of a portion of the expandable sheath of FIGS. 1 and 2.

FIG. 13A is a magnified view of a portion of the expandable sheath of FIGS. 1 and 2 with the outer layer removed for purposes of illustration.

FIG. 13B is a magnified view of a portion of the braided layer of the sheath of FIGS. 1 and 2.

FIG. 14 is a magnified view of a portion of the expandable sheath of FIGS. 1 and 2 illustrating expansion of the sheath as a prosthetic device is advanced through the sheath.

FIG. 15 is a side view of the expandable sheath of FIGS. 1 and 2.

FIG. 16 is a magnified section view of the sheath of FIG. 15 along section line 16-16.

FIG. 17 is cross sectional view of the unexpanded sheath of FIG. 16 along section line 17-17.

FIG. 18 is cross sectional view of the unexpanded sheath of FIG. 15 along section line 18-18.

FIG. 19 is cross sectional view of the unexpanded sheath of FIG. 15 along section line 19-19.

FIG. 20 is cross sectional view of the expanded sheath of FIG. 15 along section line 19-19.

FIG. 21 is a side view of the expandable sheath of FIGS. 1 and 2.

FIG. 22 is a cross section view of the unexpanded sheath of FIG. 21 along section line 22-22.

FIG. 23 is a cross sectional view of the expanded sheath of FIG. 21 along section line 22-22.

FIG. 24 is a side view of an example sheath system.

FIG. 25 is top view of an example sheath system included in a corresponding packaging tray.

### DETAILED DESCRIPTION

The following description of certain examples of the inventive concepts should not be used to limit the scope of the claims. Other examples, features, aspects, implementations, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain aspects, advantages, and novel features of the implementations of this disclosure are described herein. The described methods, systems, and apparatus should not be construed as limiting in any way. Instead, the present disclosure is directed toward all novel and nonobvious features and implementations of the various disclosed implementations, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Features, integers, characteristics, compounds, chemical moieties, or groups described in conjunction with a particular aspect or example of the present disclosure are to be understood to be applicable to any other aspect or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The present disclosure is not restricted to the details of any foregoing implementations. The present disclosure extends to any novel one, or any novel combination, of the features disclosed in this specification (including any accompanying claims, abstract, and drawings), or to any novel one, or any novel combination, of the steps of any method or process so disclosed.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

The terms “proximal” and “distal” as used herein refer to regions of a sheath, catheter, or delivery assembly. “Proximal” means that region closest to handle of the device, while “distal” means that region farthest away from the handle of the device.

“Axially” or “axial” as used herein refers to a direction along the longitudinal axis of the sheath.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

Disclosed implementations of an expandable sheath can minimize trauma to the vessel by allowing for temporary expansion of a portion of the introducer sheath to accommodate the delivery system, followed by a return to the original diameter once the device passes through. Disclosed implementations of the introducer sheath prevent the introducer from separating from the sheath during insertion by locking the proximal hub of the introducer to the proximal hub of the sheath. Fixing the introducer and the sheath prevents the introducer from moving backward during insertion, thereby maintaining a snug fit and smooth transition between the introducer and the distal end of the sheath. Furthermore, present implementations can reduce the length of time a procedure takes, as well as reduce the risk of a longitudinal or radial vessel tear, or plaque dislodgement because only one sheath is required, rather than several different sizes of sheaths. Implementations of the present expandable sheath can avoid the need for multiple insertions for the dilation of the vessel.

Example expandable introducer sheaths are disclosed, for example, in U.S. Pat. No. 8,690,936, entitled “Expandable Sheath for Introducing an Endovascular Delivery Device into a Body,” U.S. Pat. No. 8,790,387, entitled “Expandable Sheath for Introducing an Endovascular Delivery Device into a Body,” U.S. Pat. No. 10,639,152, entitled “Expandable Sheath and Methods of Using the Same,” U.S. Pat. No. 10,792,471, entitled “Expandable Sheath,” U.S. patent application Ser. No. 16/407,057, entitled “Expandable Sheath with Elastomeric Cross Sectional Portions,” U.S. Pat. No. 10,327,896, entitled “Expandable Sheath with Elastomeric Cross Sectional Portions,” U.S. Pat. No. 11,273,062, entitled “Expandable Sheath,” Application No. PCT/US2021/019514, entitled “Expandable sheath for introducing an endovascular delivery device in to a body,” Application No. PCT/US2021/031227, entitled “Expandable sheath for introducing an endovascular delivery device into a body,” Application No. PCT/US2021/031275, entitled “Expandable sheath for introducing an endovascular delivery device into a body,” U.S. application Ser. No. 17/113,268, entitled “Expandable Sheath and Method of Using the Same,” Application No. PCT/US2021/058247, entitled “Self-Expanding, Two Component Sheath,” Application No. PCT/US2022/012785, entitled “Expandable Sheath,” U.S. Pat. No. 11,051,939, entitled “Active Introducer Sheath System,” Application No. PCT/US2022/012684, entitled “Introducer with Sheath Tip Expander,” U.S. application Ser. No. 17/078,556, entitled “Advanced Sheath Patterns,” Application No. PCT/US2021/025038, entitled “Low temperature hydrophilic adhesive for use in expandable sheath for introducing an endovascular delivery device into a body,” Application No. PCT/US2021/050006, entitled “Expandable Sheath Including Reversable Bayonet Locking Hub,” U.S. Provisional Application No. 63/280,251, entitled “Expandable Sheath Gasket to Provide Hemostasis,” U.S. Provisional Application No. 63/530,144, entitled “Introducer/Dilator with Folded Balloon,” and U.S. Provisional Application No. 63/502,907, entitled “Lead Screw Driven Sheath Dilator,” the disclosures of which are herein incorporated by reference.

Elongate introducer sheaths disclosed herein are particularly suitable for delivery of implants in the form of implantable heart valves, such as balloon-expandable implantable heart valves. Balloon-expandable implantable heart valves are well-known and will not be described in detail here. An example of such an implantable heart valve is described in U.S. Pat. Nos. 5,411,552, and 9,393,110, both of which are hereby incorporated by reference. The expandable introducer sheaths disclosed herein may also be used to deliver other types of implantable medical device, such as self-expanding and mechanically expanding implantable heart valves, stents or filters. Beyond transcatheter heart valves, the introducer sheath system can be useful for other types of minimally invasive surgery, such as any surgery requiring introduction of an apparatus into a subject's vessel. For example, the introducer sheath system can be used to introduce other types of delivery apparatus for placing various types of intraluminal devices (for example, stents, stented grafts, balloon catheters for angioplasty procedures, etc.) into many types of vascular and non-vascular body lumens (for example, veins, arteries, esophagus, ducts of the biliary tree, intestine, urethra, fallopian tube, other endocrine or exocrine ducts, etc.). The term “implantable” as used herein is broadly defined to mean anything—prosthetic or not—that is delivered to a site within a body. A diagnostic device, for example, may be an implantable.

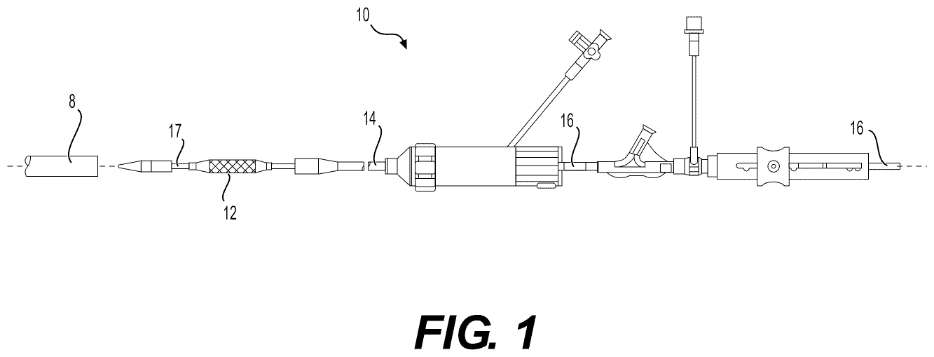

FIG. 1 illustrates an exemplary sheath 8 in use with a representative delivery apparatus 10, for delivering an implant 12, or other type of implantable (for example, tissue heart valve), to a patient. The delivery apparatus 10 can include a steerable guide catheter 14 (also referred to as a flex catheter) and a balloon catheter 16 extending through the guide catheter 14, and a nose catheter 15 extending through the balloon catheter 16. The guide catheter 14, balloon catheter 16, and nose catheter 15 in the illustrated example are adapted to slide longitudinally relative to each other to facilitate delivery and positioning of the implant 12 at an implantation site in a patient's body as described in detail herein. It is contemplated that the sheath 8 can be used with any type of elongated delivery apparatus used for implanting balloon-expandable prosthetic valves, self-expanding prosthetic valves, and other prosthetic devices.

As described in more detail herein, in general, the sheath 8 comprises an elongate expandable tube that, in use, is inserted into a vessel (for example, transfemoral vessel, femoral artery, iliac artery) by passing through the skin of patient, such that the distal end of the sheath 8 is inserted into the vessel. The sheath 8 includes a hemostasis valve and/or sealing features at the proximal end of the sheath, for example, in the sheath hub 20, that provide hemostasis and prevents blood leakage from the patient through the sheath 8. The sheath 8, including an introducer 6, is advanced into the patient's vasculature. Once positioned the introducer 6 is removed and the delivery apparatus 10 is inserted into/through the sheath 8, and the prosthetic device (implant 12) then be delivered and implanted within patient.

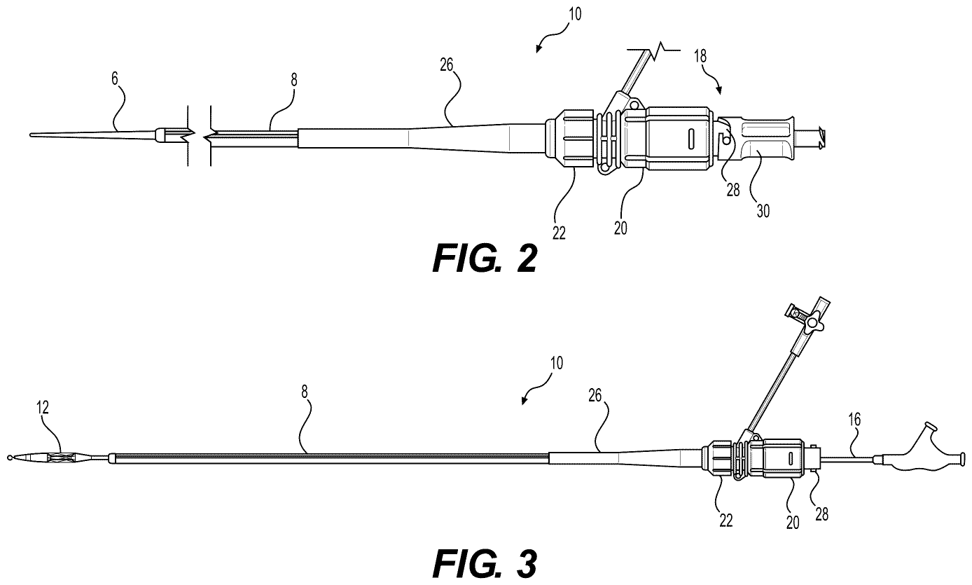

As provided in FIGS. 2 and 3, the introducer device/sheath assembly includes a sheath hub 20 at a proximal end of the device and an expandable sheath 8 extending distally from the sheath hub 20. The sheath 8 is coupled to the sheath hub 20 which in turn is removably coupled to an optional sheath locking system 18. The sheath locking system 18 allows the introducer 6, or other device desired to be removably couped (axially and rotatably) to the sheath 8.

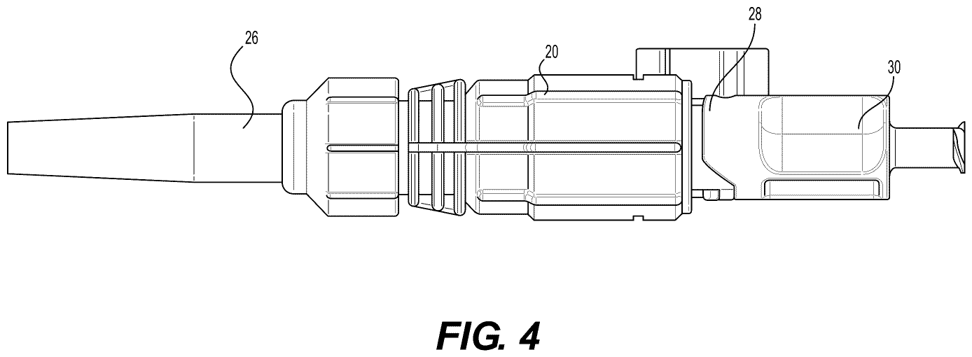

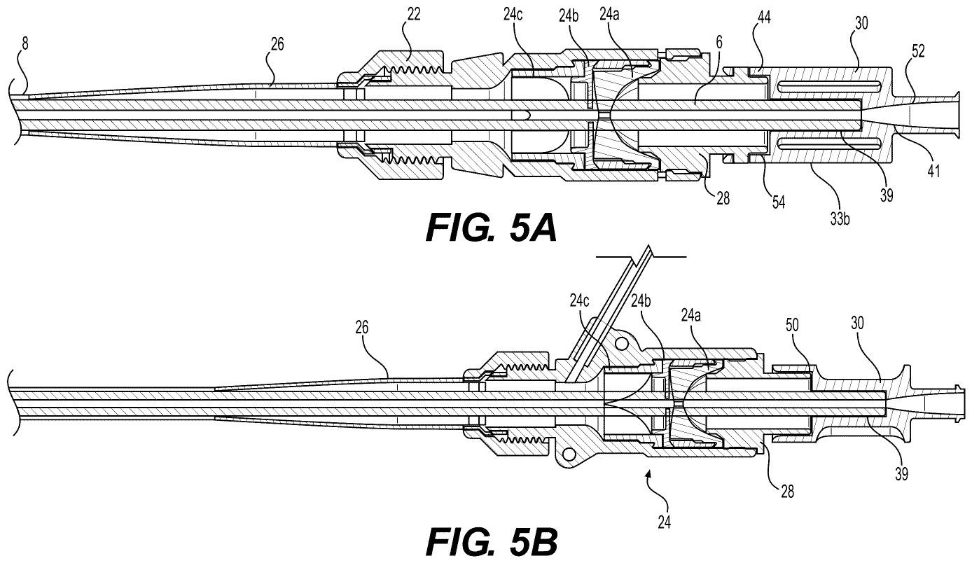

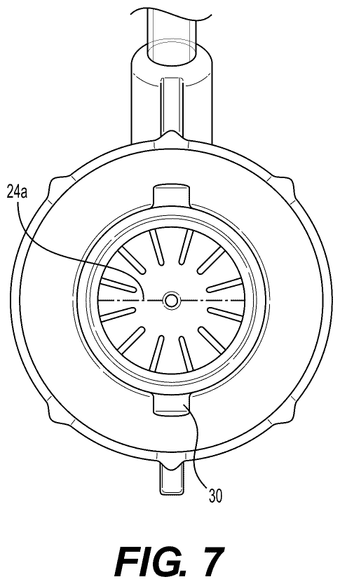

As illustrated in FIGS. 2-6, the sheath hub 20 can optionally function as a handle for the device. Sheath hub 20 also provides a housing for necessary seal assemblies and an access point for a secondary lumen (for example, fluid lumen) in fluid communication with the central lumen of the sheath hub 20. In some examples, the seal assembly 24, as described herein and as shown in FIGS. 5A, 5B, and 7, is included in the sheath hub 20. The seal assembly 24 includes a proximal seal 24a, an intermediate seal 24b, and a distal seal 24c. When assembled, the introducer 6 passes through the seal assembly and extends distal of the sheath 8. The proximal seal 24a, the intermediate seal 24b, and the distal seal 24c are each formed to prevent unwanted fluid from advancing in the proximal direction through the sheath hub 20 and proximal of the seal assembly 24. Each of the seals are openable and closable to provide pressure variation to affect the desired fluid flow from a physician or technician.

In some implementations, the distal end of the sheath hub 20 includes threads 21 for coupling to a threaded sheath hub cap 22. The sheath 8 is provided between the sheath hub 20 and the sheath hub cap 22 such that coupling the sheath hub cap 22 to the sheath hub 20 fixes the sheath 8 to the sheath hub 20. The sheath hub cap 22 is a cylindrical cap having a cap body having a proximal end and a distal end and defining a central lumen extending longitudinally between the proximal end and the distal end. The sheath hub cap 22 has a larger diameter at its proximal end than at its distal end.

In some implementations, the sheath hub 20 further includes receiving slots 48 for coupling the sheath locking system 18, particularly the locking sleeve 28, to the sheath hub 20. In some implementations, example receiving slots 48 comprise openings which extend around a portion of the diameter of the sheath hub 20 and are sized and configured to accept the interference diameters 66 of the locking sleeve 28. Coupling between the receiving slots 48 and the interference diameters 66 axially and rotationally fixes the locking sleeve 28 and the sheath hub 20 relative to each other.

FIG. 2 illustrates the sheath 8 of FIG. 1 including an optional sheath locking system 18 for preventing axial and rotational translation of the introducer 6 with respect to the sheath 8. Example locking systems are disclosed in PCT/US2021/050006, entitled “Expandable Sheath Including Reverse Bayonet Locking Hub,” the disclosure of which is incorporated herein by reference. It is contemplated that the locking system 18 disclosed herein can also optionally be used to couple the sheath 8/sheath hub 20 with other delivery system components, catheters, dilators, etc. including the same mating features.

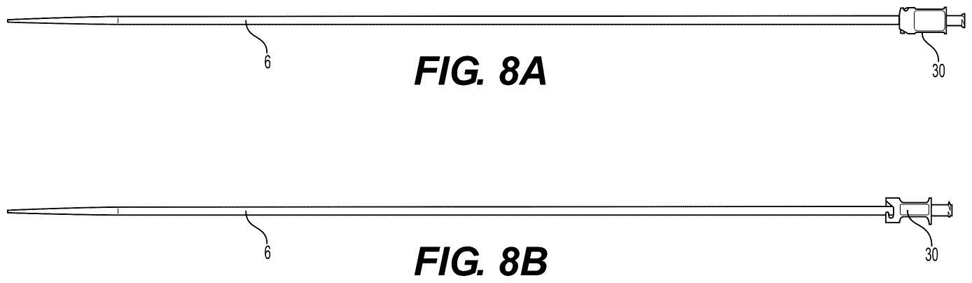

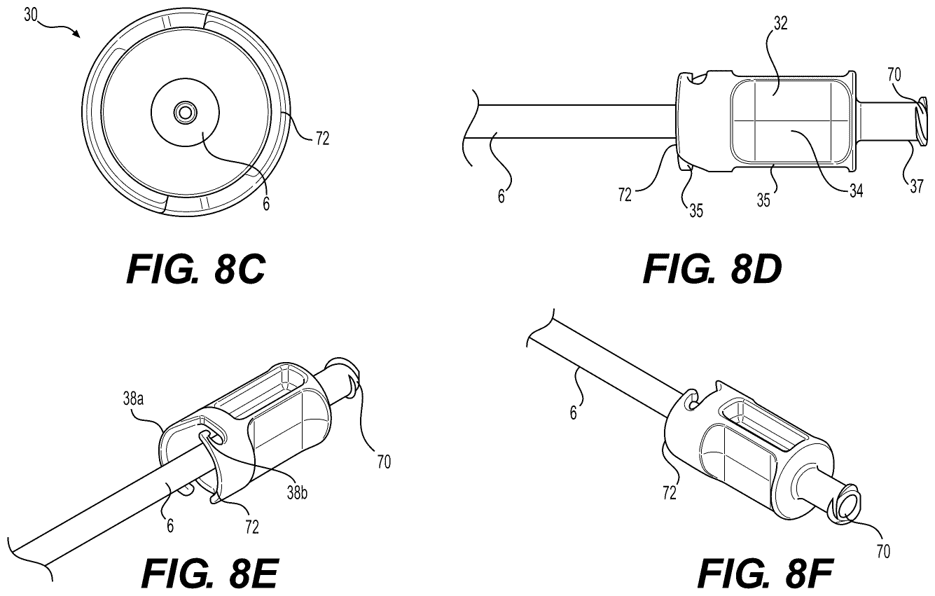

As described herein, in some implementations, the sheath locking system 18 is used to fix the introducer 6 with respect to the sheath 8 during insertion without requiring a physician or technician to hold the introducer 6 and the sheath 8 in place at the distal end. As illustrated in FIGS. 8A-8B, the sheath locking system 18 includes a locking sleeve 28 and an introducer locking hub 30 (including corresponding introducer 6). In some implementations, the locking sleeve 28 is coupled to the sheath 8 via the sheath hub 20. The locking sleeve 28 engages the introducer locking hub 30 and is moveable between a locked and unlocked position, thereby fixing the position of the introducer 6 and the sheath 8 and preventing movement therebetween, particularly during insertion into the patient. As will be described in more detail herein, the sheath locking system 18 can be used to keep the introducer 6 from separating from the sheath 8 and prevent gaps from forming that can cause patient abrasions and unintended fluid flow between the introducer 6 and the sheath 8 during insertion.

FIGS. 2, 5A-5B and 6, and illustrate the sheath locking sleeve 28 coupled to the introducer locking hub 30 and the sheath hub 20. As will be described in more detail herein, in some implementations, the locking sleeve 28 includes a guide 31 that engages a locking channel 38 provided on the introducer locking hub 30. The guide 31 is movable within the locking channel 38 between an unlocked position, where the sheath locking sleeve 28 is rotationally and axially movable with respect to the introducer locking hub 30, and a locked position (shown in FIG. 2), where the locking sleeve 28 is axially fixed with respect to the introducer locking hub 30.

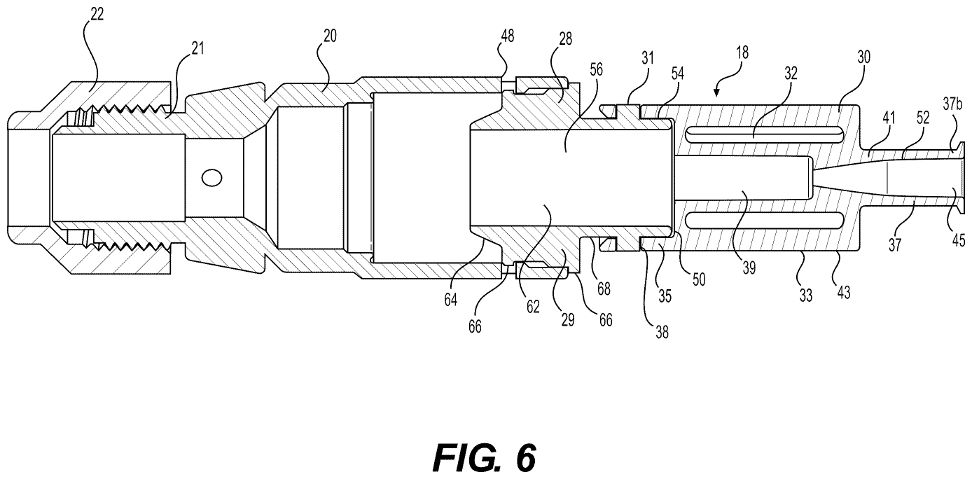

The locking sleeve 28 is illustrated, for example, in FIGS. 10A-10F. The locking sleeve 28 includes an elongated sleeve body 29 with a central lumen 56 extending longitudinally between the proximal end 58 and distal end 60 of the sleeve body 29. As provided in the cross-sectional view of the sheath locking system 18 illustrated in FIG. 6, the central lumen 56 defines a generally cylindrical inner surface 62 of the sheath locking sleeve 28. In some implementations, the central lumen 56 has a diameter of at least 0.3′. In some examples, the diameter ranges between 0.3′ and 0.6′. Preferably, the diameter is about 0.40′. The distal end 60 of the sleeve body 29 optionally has a frustoconical outer surface 64 that tapers about the distal end 60 to help with positioning the locking sleeve 28 within the sheath hub 20 and abutting the seal assembly 24, as shown in FIGS. 5A and 5B). In some implementations, the locking sleeve 28 also has a plurality of interference diameters 66 that extend radially from the outer surface of the sleeve body 29 around all or a portion of the circumference of the locking sleeve 28. As illustrated in FIG. 5A and FIG. 6, the distally positioned interference diameters 66 are sized and configured to engage corresponding recesses and/or slots 48 provided in the sheath hub 20 for securing the locking sleeve 28 to the sheath hub 20. In some implementations, as illustrated in FIG. 6, the proximally positioned interference diameter 66 optionally seats against the proximal end of the sheath hub 20.

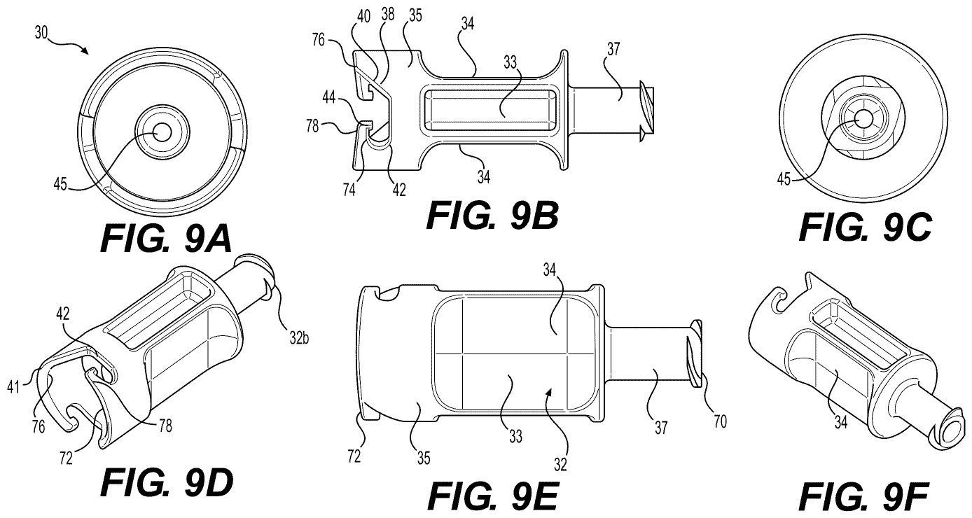

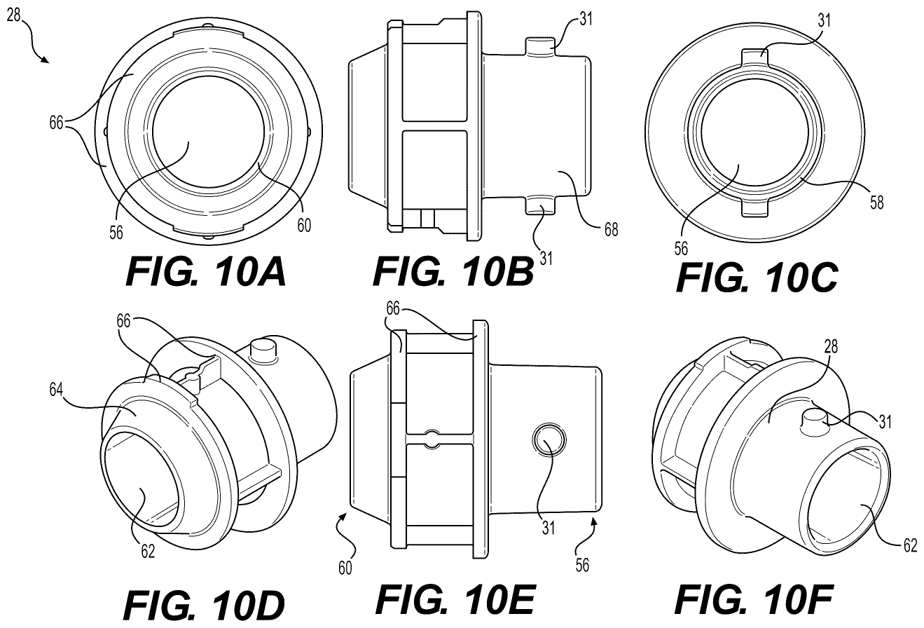

As illustrated in FIG. 10B, the locking sleeve 28 includes a guide 31 projecting from the outer surface 68 of the locking sleeve 28. As described herein, the guide 31 is size to configured to engage a corresponding shaped locking channel 38 in the introducer locking hub 30, illustrated in FIG. 9B. The guide 31 extends radially from the outer surface 68 and at least partially around the circumference of the outer surface 68. As provided in FIG. 6, in some implementations, when the locking sleeve 28 and introduce your locking hub 30 are coupled, the top surface of the guide 31 does not extend beyond the outer surface of the introducer locking hub 30. For example, the height of the guide 31 can optionally be sized correspond with the wall thickness of the introducer locking hub 30 proximate the guide when the sheath locking sleeve 28 and the introducer locking hub 30 are coupled. In another example, the top surface of the guide 31 can optionally be recessed with respect to the outer surface of the introducer locking hub 30. That is, the height of the guide 31 is less than the wall thickness of the introducer locking hub 30. In other examples, the height of the guide 31 can optionally be greater than a wall thickness of the introducer locking hub 30 such that the top surface of the guide 31 extends beyond the outer surface of the introducer locking hub 30 when the sheath locking sleeve 28 and the introducer locking hub 30 are coupled. In some examples, the height/axial length of the guide 31 is between about 0.050′ and about 0.10.′ In some examples that height/axial length of the guide 31 is about 0.075′.

As illustrated in FIGS. 10D-10F, the guide 31 is optionally defined as a cylindrically shaped projection. However, it is contemplated that the guide 31 may have any other regular or irregular shape that would facilitate movement of the guide 31 within the locking channel 38 of the introducer locking hub 30. For example, the guide 31 may optionally have an elongated hexagon shape. The guide 31 can have a diameter/width ranging from about 0.05′ to about 0.20′. Preferably the guide 31 has a diameter/width of about 0.100′.

In general, the locking sleeve 28 can optionally be formed from polycarbonate. In other implementations, the locking sleeve 28 can be formed from rigid plastic, or any other material suitable for providing a strong locking connector for an introducer 6 including, for example, a metal, composite, or other suitable material.

FIGS. 2-6 illustrate the introducer locking hub 30 coupled to the locking sleeve 28. FIGS. 8A-8F show the introducer locking hub 30 coupled to the introducer 6. FIGS. 9A-9F provide multiple views of the introducer locking hub 30. As described herein, the introducer 6 can be fixedly coupled to the introducer locking hub 30. For example, in some implementations, the introducer locking hub 30 couples with the locking sleeve 28 to fix the position the introducer 6 (axially and rotationally) with respect to the locking sleeve 28/sheath 8. Each of the introducer 6 and introducer locking hub 30 are described in more detail herein.

FIGS. 8A-8F illustrate the introducer locking hub 30 with an example introducer 6 coupled thereto. As provided in the cross-section views of FIGS. 5A and 5B, the introducer 6 is coupled to the introducer locking hub 30 and extends beyond the distal end of the introducer locking hub 30 body. When coupled to the sheath hub 20, the introducer 6 extends through the central lumen 56 of the sheath locking sleeve 28, the sheath hub 20 and the central lumen of the sheath 8. As described herein, the sheath 8 generally comprises a radially expandable tubular structure. Passage of the introducer 6 through the sheath 8 positioned in the patient's vasculature causes sheath 8 to expand and results in a corresponding radial expansion of the blood vessel to about the diameter of the sheath 8. That is, the diameter of the central lumen of the sheath 8 generally corresponds to the outer diameter of the introducer 6 such that the introducer 6 provides a mechanism to expand/pre-dilate a patient's vessel to accept the medical device/implant 12.

As provided in FIGS. 8A-8F, the introducer 6 is formed as an elongate body and optionally includes a central lumen extending therethrough. As shown in FIGS. 5A and 5B, when assembled, the central lumen of the introducer 6 is aligned with the central lumens of the introducer locking hub 30, the sheath hub 20, and the sheath 8. In some implementations, as provided in FIGS. 5A and 5B, the introducer 6 is received within a recessed opening 39 provided on an interior surface of the introducer locking hub 30, where the recessed opening 39 axially aligned with the central lumen 45 of the introducer locking hub 30. In some implementations, the introducer 6 is fixedly or releasably coupled to the introducer locking hub 30 at the recessed opening 39. In some examples, the introducer 6 is fixedly coupled to the introducer locking hub 30 at the recessed opening 39. In some implementations, the introducer 6 has a diameter corresponding to, or less than, the diameter of the recessed opening 39. The introducer 6 can be coupled to the recessed opening 39 of the introducer locking hub 30 by at least one of a press fit, an interference fit, a snap fit, a mechanical fastener, a chemical fastener (for example, an adhesive), a weld, a thermal process, and/or any other suitable coupling process known in the art.

As described herein, the introducer 6 optionally includes a central lumen that aligns with the central lumen 45 of the introducer locking hub 30. When coupled, this joined lumen allows for the passage of surgical equipment and/or medical devices to the treatment site (for example, a guide wire). In an example system, and as provided in FIGS. 5A and 5B, the central lumen of the introducer 6 has a diameter corresponding to at least a portion of the diameter of the central lumen 45 of the introducer locking hub 30. In general, the corresponding diameter portion is adjacent the distal end of the central lumen 45. In other examples, the diameter of the central lumen 45 at the distal end of the introducer locking hub 30 is slightly larger than the diameter of the central lumen passing through the introducer 6. The central lumen 45 can optionally define a decreasing tapered portion 41 extending between the proximal end and the distal end of the introducer locking hub 30, as shown in FIG. 6. The corresponding diameter portion and decreasing tapered portion 41 allows for smooth transition and delivery of surgical equipment and/or medical device through the introducer locking hub 30 and into the central lumen of the introducer 6.

As illustrated in FIGS. 9A-9F, the introducer locking hub 30 includes a hub body 32 having a proximal end 70 and a distal end 72 and optionally defines a central lumen 45 extending therethrough. The hub body 32 has a first (middle) portion 33, a second (distal) portion 35 which extends distally from the first portion 33, and a third (proximal) portion 37 which extends proximally from the first portion 33. The first portion 33 can optionally include the cylindrically-shaped recessed opening 39 for receiving and retaining the introducer 6 and an outer surface 43. In some examples, the recessed opening 39 has a diameter ranging between 0.15′ and about 0.25′. In some examples, the recessed opening 39 has a diameter ranging between 0.17′ and about 0.20′. In some examples, the recessed opening has a diameter of about 0.194′.

In some implementations, the third (proximal) portion 37 of the introducer locking hub 30 includes the decreasing tapered portion 41 of the central lumen 45. As illustrated in FIG. 6, the decreasing taper portion 41 defines a frustoconical shape with decreasing taper/diameter in a direction extending from the proximal toward the distal end of the sheath 8. It is contemplated that the tapered portion 41 has a minimum diameter of about 0.007′ and a maximum diameter of about 0.194′.

As illustrated in FIGS. 5A and 5B, when coupled, the central lumen 56 of the locking sleeve 28 is aligned with the central lumen 45 of the introducer locking hub 30. In some examples, the central lumen 56 of the locking sleeve 28 is coaxial with the central lumen 45 of the introducer locking hub 30. When coupled, the proximal end of the locking sleeve 28 is received within the central lumen 45 of the introducer locking hub 30. As such, in some implementations, the proximal end surface of the locking sleeve 28 is located adjacent a shoulder 50 provided on an inner surface of the central lumen 45 of the introducer locking hub 30. As illustrated in FIGS. 5A and 5B, the central lumen 45 of the introducer locking hub 30 includes a first portion 52 and a second portion 54. The first portion 52 is located adjacent the proximal end of the introducer locking hub 30 and defines a first diameter, and the second portion 54 is located adjacent the distal end of the introducer locking hub 30 and defines a second, larger, diameter. The recessed opening 39 of the introducer locking hub 30 can optionally be considered a component of either the first portion 52 of the central lumen 45, or a separate component of the central lumen 45 located between the first (proximal) portion 52 and the second (distal) portion 54. When the locking sleeve 28 and introducer locking hub 30 are coupled, at least a portion of the sleeve body 29 of the sheath locking sleeve 28 is received within the second portion 54 (larger diameter portion) of the central lumen 45 of the introducer locking hub 30. In some implementations, the central lumen 56 of the sheath locking sleeve 28 is aligned with the central lumen 45 of the introducer locking hub 30 such that they are co-axial and form a smooth inner surface along the combined central lumens of the introducer locking hub 30 and the sheath locking sleeve 28.

As described herein, the locking sleeve 28 can be coupled to the introducer locking hub 30 via engagement between the guide 31 provided on the locking sleeve 28 and the locking channel 38 provided in the introducer locking hub 30. As illustrated in FIGS. 9A-9F, the introducer locking hub 30 optionally includes two locking channels 38. However, it is contemplated that the introducer locking hub 30 can include one locking channel 38 or more than two locking channels 38. The locking channel 38 can optionally be formed a recess or groove in a surface of the introducer locking hub 30, or as a slotted opening, clip, or any other feature capable of receiving and securing the guide 31/locking sleeve 28 with the introducer locking hub 30. Illustrated in FIG. 9B, the locking channels 38 provide an interface to secure the sheath locking sleeve 28 to the introducer locking hub 30 and ensure a fixed axial position between the introducer 6 and the sheath 8.

As illustrated in FIG. 9B, the locking channel 38 is formed on or proximal the distal end of the introducer locking hub 30. In some implementations, the locking channel 38 includes an opening on the distal end surface of the introducer locking hub 30. The opening to the locking channel 38 and extend to an angled guide portion 40 that transitions/extends to a locking portion 42. In some implementations, the guide portion 40 is configured to direct the guide 31 provided on the locking sleeve 28 in an axial and/or circumferential direction along the side wall of the guide portion 40 towards the locking portion 42 upon rotation of the introducer locking hub 30 and/or the sheath locking sleeve 28. In some implementations, the locking portion 42 is configured to securely engage the guide 31, fixing the axial position of the introducer locking hub 30 with respect to the sheath locking sleeve 28. As illustrated in FIG. 9B, the guide portion 40 of the locking channel 38 extends from the distal end of the introducer locking hub 30 axially towards the proximal end of the introducer locking hub 30 and also circumferentially around the introducer locking hub 30. For example, in some implementations, the guide portion 40 of the locking channel 38 can be described as extending helically around/along a length of the introducer locking hub 30 or on an angle from the distal end of the introducer locking hub 30.

As illustrated in FIGS. 9B and 9D, the locking portion 42 of the locking channel 38 extends from the end of the guide portion 40. In some implementations, the locking portion 42 extends at an angle from the end of the guide portion 40. As provided in FIG. 9B, the angle between the centerline of the guide portion 40 and the centerline of the locking portion 42 is greater than 90-degrees. In another example, the angle between the centerline of the guide portion 40 and the centerline of the locking portion 42 is about 120-degrees. In an example system, the locking portion 42 extends around a portion of the circumference of the introducer locking hub 30. The locking portion 42 can optionally extend generally parallel to the distal end surface of the introducer locking hub 30. In some implementations, the locking portion 42 can optionally extend circumferentially around the introducer locking hub 30 in a direction generally perpendicular to the longitudinal axis of the introducer locking hub 30 and/or introducer 6. In an example system, the length of the guide portion 40 (measured along its centerline) is greater than the length of the locking portion 42 (measured along its centerline). In another example, the length of the guide portion 40 equals or is less than the length of the locking portion 42.

The locking portion 42 can optionally include a catch 44 for securing the guide 31 within the locking portion 42 of the locking channel 38 and forming a partial barrier for maintaining the guide 31 within the locking portion 42. As illustrated in FIG. 9B, the catch 44 includes a projection that extends from the side wall 74 of the locking portion 42 and is sized and configured to releasably secure the guide 31 within the locking channel 38. In some implementations, the catch 44 extends from the side wall 42a of the locking portion 42 in a proximal direction towards the center line of the locking portion 42 and has a height sufficient to retain the guide 31 between the catch 44 and the end of the locking portion 42.

In some implementations, the distal end surface 72 of the introducer locking hub 30 optionally includes features for biasing the guide 31 towards and/or into the locking channel 38. For example, in some implementations, the distal end of the introducer locking hub 30 can include a tapered surface angled toward the opening of the locking channel 38. For example, as illustrated in FIG. 9B, the distal end 72 of the introducer locking hub 30 includes a first tapered surface 76 angled towards the leading edge of the opening of the locking channel 38 and a second tapered surface 78 angled towards the trailing edge of the opening of the locking channel 38. The angle of the first tapered surface 76 and second tapered surface 78 help to urge the guide 31 in a proximal direction and into the locking channel 38.

In use, engagement between the guide 31 and the guide portion 40 of the locking channel 38 is configured to bias the locking sleeve 28 in a proximal direction toward the proximal end 70 of the introducer locking hub 30 (towards the locked position) when the sheath locking sleeve 28 is rotated in a first axial direction. In this direction the guide 31 advances toward the locking portion 42 of the locking channel 38 into the locked position. Alternatively, when the sheath locking sleeve 28 is rotated in a second (opposite) axial direction, engagement between the guide 31 and the locking portion 42 of the locking channel 38 is configured to bias the locking sleeve 28 in a distal axial direction toward the distal end of the introducer locking hub 30 (towards an unlocked position). In the second direction, the guide 31 moves away from the locking portion 42 of the locking channel 38, to/toward the unlocked position. For example, when the guide 31 is in the locked position and retained within the locking portion 42 by catch 44, rotation in the second direction causes the guide 31 to bias against the catch 44 overcoming the oppositional forces of the catch 44. Further rotation in the second direction causes the guide 31 to move beyond the catch 44 and into/through the guide portion 40, from the locked to the unlocked position.

As illustrated in FIGS. 8A-9F, the outer surface of the introducer locking hub body 32 optionally includes gripping features and/or surfaces for a physician or technician to use when manipulating the introducer locking hub 30. As provided in FIG. 9B, the introducer locking hub body 32 can optionally include two recessed gripping surfaces 34 on opposite sides of the longitudinal axis of the introducer locking hub 30. In some implementations, when the introducer locking hub 30 is viewed from the side, the gripping surfaces 34 define a dog-bone/barbell-shaped hub body 32, i.e., a shape having a smaller diameter/width center portion and larger diameter/width end portions. In an example system, the gripping surfaces 34 are optionally provided along at least 40% of the length of the introducer locking hub body 32. In another example, the gripping surfaces 34 are provided along at least 50% of the length of the introducer locking hub body 32.

In general, the introducer locking hub 30 can be formed from polycarbonate. In other implementations, the introducer locking hub 30 can be formed from rigid plastic, or any other material suitable for providing a locking mechanism for an introducer 6 including, for example, a metal, composite, or other suitable material.

As described herein, the introducer device/sheath assembly includes an expandable sheath 8 extending distally from the sheath hub 20. The expandable sheath 8 has a central lumen to guide passage of the delivery apparatus 10 for the medical device/implant 12 (prosthetic heart valve). In some implementations, the introducer device/sheath assembly need not include the sheath hub 20. For example, the sheath 8 can be an integral part of a component of the sheath assembly, such as the guide catheter.

As described herein, the expandable sheath 8 can have a natural, unexpanded outer diameter that will expand locally upon passage of the medical device. For example, the expandable sheath 8 can be formed from a highly elastomeric material where vessel dilation is performed by the passing prosthetic device.

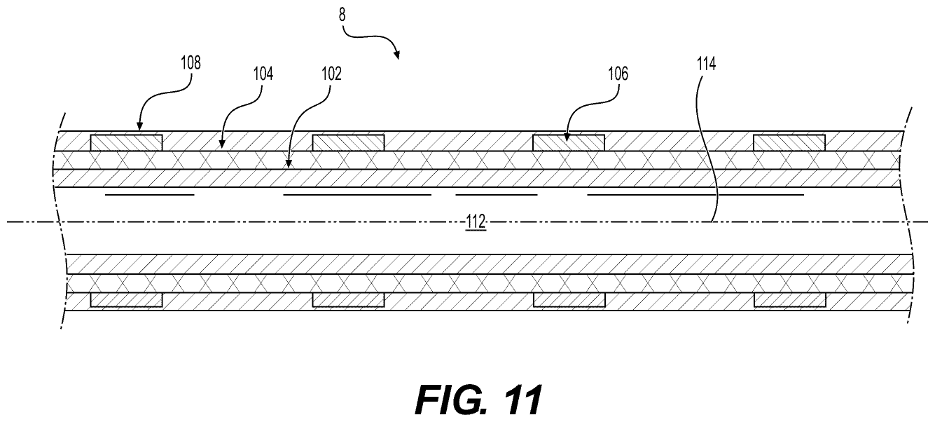

In some implementations, the expandable sheath 8 can comprise a plurality of coaxial layers extending along at least a portion of the length of the sheath 8. The structure of the coaxial layers is described in more detail herein with respect to FIGS. 11-23. Example expandable sheaths including coaxial layers are described, for example, in U.S. patent application Ser. No. 16/378,417, entitled “Expandable Sheath,” and U.S. patent application Ser. No. 17/716,882, entitled “Expandable Sheath,” the disclosures of which are herein incorporated by reference.

Various implementations of the coaxial layered structure of the sheath 8 are described herein. For example, in reference to the example sheath 8 illustrated in FIGS. 11-14, the expandable sheath 8 can include a number of layers including an inner layer 102 (also referred to as an inner layer), a second layer 104 disposed around and radially outward of the inner layer 102. In some implementations, the sheath 8 includes a third layer 106 disposed around and radially outward of the second layer 104, and a fourth outer layer 108 (also referred to as an outer layer) disposed around and radially outward of the third layer 106. In the illustrated configuration, the inner layer 102 can define the lumen 112 of the sheath extending along a central axis 114 through which the delivery apparatus travels into the patient's vessel to deliver, remove, repair, and/or replace a prosthetic device, moving in a direction along the longitudinal axis of the sheath 8.

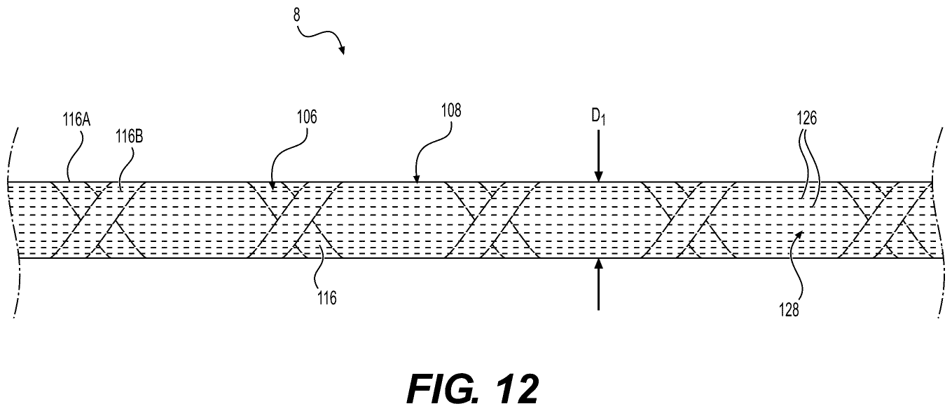

Referring to FIG. 12, when the sheath 8 is in an unexpanded state, various layers of the sheath, for example, the inner layer 102 and/or the outer layer 108, can optionally form longitudinally-extending folds or creases such that the surface of the sheath comprises a plurality of ridges 126 (also referred to herein as “folds”). The ridges 126 can be circumferentially spaced apart from each other by longitudinally-extending valleys 128. When the sheath 8 expands beyond its natural diameter D₁, the ridges 126 and the valleys 128 can level out or be taken up as the surface radially expands and the circumference so the sheath 8 increases, as further described herein. When the sheath 8 collapses back to its natural diameter, the ridges 126 and valleys 128 can reform.

In some implementations, the inner layer 102 and/or the outer layer 108 can comprise a relatively thin layer of polymeric material. For example, in some implementations the thickness of the inner layer 102 can be from 0.01 mm to 0.5 mm, 0.02 mm to 0.4 mm, or 0.03 mm to 0.25 mm. In some implementations, the thickness of the outer layer 108 can be from 0.01 mm to 0.5 mm, 0.02 mm to 0.4 mm, or 0.03 mm to 0.25 mm.

In some examples, the inner layer 102 and/or the outer layer 108 can comprise a lubricious, low-friction, and/or relatively non-elastic material. In particular implementations, the inner layer 102 and/or the outer layer 108 can comprise a polymeric material having a modulus of elasticity of 400 MPa or greater. Exemplary materials can include ultra-high-molecular-weight polyethylene (UHMWPE) (for example, Dyneema®), high-molecular-weight polyethylene (HMWPE), or polyether ether ketone (PEEK). With regard to the inner layer 102 in particular, such low coefficient of friction materials can facilitate passage of the prosthetic device through the lumen 112. Other suitable materials for the inner layer 102 and outer layer 108 can include polyimide, polytetrafluoroethylene (PTFE), expanded polytetrafluoroethylene (ePTFE), ethylene tetrafluoroethylene (ETFE), nylon, polyethylene, polyamide, polyether block amide (for example, Pebax), and/or combinations of any of the herein. Some implementations the sheath 8 can optionally include a lubricious liner on the inner surface of the inner layer 102. Examples of suitable lubricious liners include materials that can further reduce the coefficient of friction of the inner layer 102, such as PTFE, polyethylene, polyvinylidine fluoride, and combinations thereof. Suitable materials for a lubricious liner also include other materials desirably having a coefficient of friction of 0.1 or less.

Additionally, some implementations of the sheath 8 can optionally include an exterior hydrophilic coating on the outer surface of the outer layer 108. Such a hydrophilic coating can facilitate insertion of the sheath 8 into a patient's vessel, reducing potential damage. Examples of suitable hydrophilic coatings include the Harmony™ Advanced Lubricity Coatings and other Advanced Hydrophilic Coatings available from SurModics, Inc., Eden Prairie, MN. DSM medical coatings (available from Koninklijke DSM N. V, Heerlen, the Netherlands), as well as other hydrophilic coatings (for example, PTFE, polyethylene, polyvinylidine fluoride), are also suitable for use with the sheath 8. Such hydrophilic coatings may also be included on the inner surface of the inner layer 102 to reduce friction between the sheath 8 and the delivery system, thereby facilitating use and improving safety. In some implementations, a hydrophobic coating, such as Perylene, may be used on the outer surface of the outer layer 108 or the inner surface of the inner layer 102 in order to reduce friction.

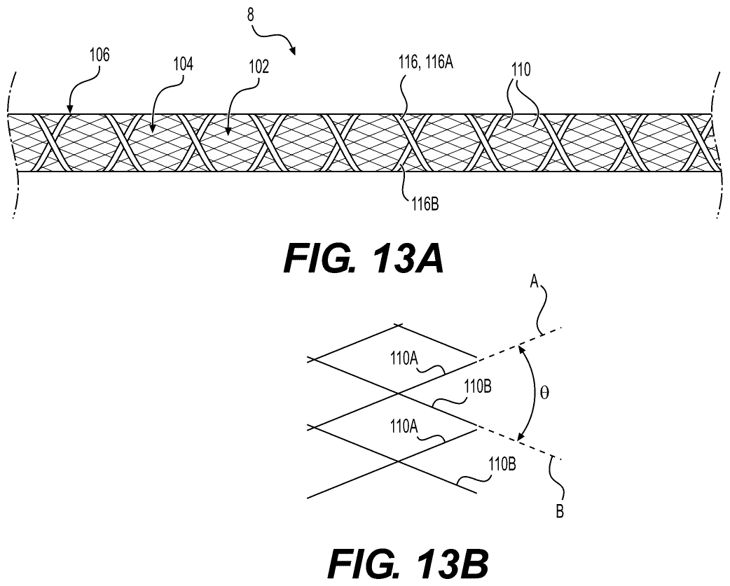

In some implementations, the second layer 104 can be a braided layer. FIGS. 13A and 13B illustrate the sheath 8 with the outer layer 108 removed to expose the elastic third layer 106. With reference to FIGS. 13A and 13B, the braided second layer 104 can comprise a plurality of members or filaments 110 (for example, metallic or synthetic wires or fibers) braided together. The braided second layer 104 can have any desired number of filaments 110, which can be oriented and braided together along any suitable number of axes. For example, with reference to FIG. 13B, the filaments 110 can include a first set of filaments 110A oriented parallel to a first axis A, and a second set of filaments 110B oriented parallel to a second axis B. The first set of filaments 110A and the second set of filaments 110B can be braided together in a biaxial braid such that filaments 110A oriented along axis A form an angle θ with the filaments 110B oriented along axis B. In some implementations, the angle θ can be from 5° to 70°, 10°to 60°, 10°to 50°, or 10° to 45°. In the illustrated example, the angle θ is 45°. In other implementations, the filaments 110 can also be oriented along three axes and braided in a triaxial braid, or oriented along any number of axes and braided in any suitable braid pattern. The braided second layer 104 can extend along substantially the entire length L of the sheath 8, or alternatively, can extend only along a portion of the length of the sheath. In particular implementations, the filaments 110 can be wires made from metal (for example, Nitinol, stainless steel, etc.), or any of various polymers or polymer composite materials, such as carbon fiber. In some implementations, the filaments 110 can be round, and can have a diameter of from 0.01 mm to 0.5 mm, 0.03 mm to 0.4 mm, or 0.05 mm to 0.25 mm. In other implementations, the filaments 110 can have a flat cross-section with dimensions of 0.01 mm×0.01 mm to 0.5 mm×0.5 mm, or 0.05 mm×0.05 mm to 0.25 mm×0.25 mm. In one aspect, filaments 110 having a flat cross-section can have dimensions of 0.1 mm×0.2 mm. However, other geometries and sizes are also suitable for some implementations. If braided wire is used, the braid density can optionally be varied along a length of the sheath 8. Some implementations have a braid density of from ten picks per inch to eighty picks per inch, and can include eight wires, sixteen wires, or up to fifty-two wires in various braid patterns. In other implementations, the second layer 104 can be laser cut from a tube, or laser-cut, stamped, punched, etc., from sheet stock and rolled into a tubular configuration. The second layer 104 can also be woven or knitted, as desired.

The third layer 106 can be a resilient, elastic layer (also referred to as an elastic material layer). In some implementations, the elastic third layer 106 can be configured to apply radially inward force to the underlying layers 102 and 104 in a radial direction (for example, toward the central axis 114 of the sheath) when the sheath 8 expands beyond its natural diameter by passage of the delivery apparatus through the sheath 8. Stated differently, the elastic third layer 106 can be configured to apply encircling/radially inward pressure to the layers of the sheath 8 beneath the elastic third layer 106 (for example, layers 102 and 104) to counteract expansion of the sheath 8. The radially inwardly directed force is sufficient to cause the sheath 8 to collapse radially back to its unexpanded state after the delivery apparatus has passed through the sheath 8.

In some implementations, the elastic third layer 106 can optionally include one or more members configured as strands, ribbons, or elastic bands 116 helically wrapped around the braided second layer 104. For example, in the illustrated aspect the elastic third layer 106 comprises two elastic bands 116A and 116B wrapped around the braided second layer 104 with opposite helicity, although the elastic third layer 106 may comprise any number of bands depending upon the desired characteristics. The elastic bands 116A and 116B can be made from, for example, any of a variety of natural or synthetic elastomers, including silicone rubber, natural rubber, any of various thermoplastic elastomers, polyurethanes such as polyurethane siloxane copolymers, urethane, plasticized polyvinyl chloride (PVC), styrenic block copolymers, polyolefin elastomers, etc. In some implementations, the elastic third layer 106 can comprise an elastomeric material having a modulus of elasticity of 200 MPa or less. In some implementations, the elastic third layer 106 can comprise a material exhibiting an elongation to break of 200% or greater, or an elongation to break of 400% or greater. The elastic third layer 106 can also take other forms, such as a tubular layer comprising an elastomeric material, a mesh, a shrinkable polymer layer such as a heat-shrink tubing layer, etc. In lieu of, or in addition to, the elastic third layer 106, the sheath 8 may also include an elastomeric or heat-shrink tubing layer around the outer layer 108. Examples of such elastomeric layers are disclosed in U.S. Publication No. 2014/0379067, U.S. Publication No. 2016/0296730, and U.S. Publication No. 2018/0008407, which are incorporated herein by reference. In other implementations, the elastic third layer 106 can also be radially outward of the polymeric outer layer 108.

In some implementations, one or both of the inner layer 102 and/or the outer layer 108 can be configured to resist axial elongation of the sheath 8 when the sheath 8 expands. More particularly, one or both of the inner layer 102 and/or the outer layer 108 can resist stretching against longitudinal forces caused by friction between a prosthetic device and the inner surface of the sheath 8 such that the length L remains substantially constant as the sheath 8 expands and contracts. As used herein with reference to the length L of the sheath 8, the term “substantially constant” means that the length L of the sheath 8 increases by not more than 1%, by not more than 5%, by not more than 10%, by not more than 15%, or by not more than 20%. Meanwhile, with reference to FIG. 13B, the filaments 110A and 110B of the braided second layer 104 can be allowed to move angularly relative to each other such that the angle θ changes as the sheath 8 expands and contracts. This, in combination with the longitudinal folds/ridges 126 in the inner layer 102 and the outer layer 108, can allow the lumen 112 of the sheath 8 to expand as a prosthetic device/implant 12 is advanced through it.

In some implementations the inner layer 102 and the outer layer 108 can be heat-bonded during the manufacturing process such that the braided second layer 104 and the elastic third layer 106 are encapsulated between the layers 102 and the outer layer 108. More specifically, in some implementations the inner layer 102 and the outer layer 108 can be adhered to each other through the spaces between the filaments 110 of the braided second layer 104 and/or the spaces between the elastic bands 116. The layers 102 and the outer layer 108 can also be bonded or adhered together at the proximal and/or distal ends of the sheath 8. In some implementations, the layers 102 and the outer layer 108 are not adhered to the filaments 110. This can allow the filaments 110 to move angularly relative to each other, and relative to the layers 102 and the outer layer 108, allowing the diameter of the braided second layer 104, and thereby the diameter of the sheath 8, to increase or decrease. As the angle θ between the filaments 110A and 110B changes, the length of the braided second layer 104 can also change. For example, as the angle θ increases, the braided second layer 104 can foreshorten, and as the angle θ decreases, the braided second layer 104 can lengthen to the extent permitted by the areas where the layers 102 and the outer layer 108 are bonded. However, because the braided second layer 104 is not adhered to the layers 102 and the outer layer 108, the change in length of the braided layer that accompanies a change in the angle θ between the filaments 110A and 110B does not result in a significant change in the length L of the sheath 8.

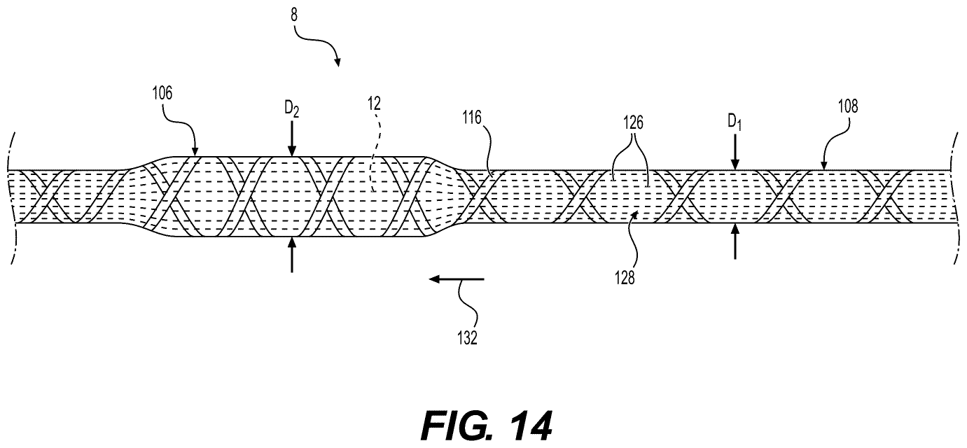

FIG. 14 illustrates radial expansion of the sheath 8 as a prosthetic device (for example, implant 12) is passed through the sheath 8 in the direction of arrow 132 (for example, distally). As the prosthetic device/implant 12 is advanced through the sheath 8, the sheath can resiliently expand to a second diameter D₂ that corresponds to a size or diameter of the prosthetic device/implant 12. As the prosthetic device/implant 12 is advanced through the sheath 8, the prosthetic device/implant 12 can apply longitudinal force to the sheath 8 in the direction of motion by virtue of the frictional contact between the prosthetic device and the inner surface of the sheath 8. However, as noted herein, in some implementations, the inner layer 102 and/or the outer layer 108 can resist axial elongation such that the length L of the sheath 8 remains constant, or substantially constant. This can reduce or prevent the braided second layer 104 from lengthening, and thereby constricting the lumen 112.

Meanwhile, the angle θ between the filaments 110A and 110B can increase as the sheath 8 expands to the second diameter D₂ to accommodate the prosthetic device/implant 12. This can cause the braided second layer 104 to foreshorten. However, in some implementations, because the filaments 110 are not engaged or adhered to the inner layer 102 or outer layer 108, the shortening of the braided second layer 104 attendant to an increase in the angle θ does not affect the overall length L of the sheath 8. Moreover, because of the longitudinally-extending folds/ridges 126 formed in the inner layer 102 and outer layer 108, the inner layer 102 and outer layer 108 can expand to the second diameter D₂ without rupturing, in spite of being relatively thin and relatively non-elastic. In this manner, the sheath 8 can resiliently expand from its natural diameter D₁ to a second diameter D₂ that is larger than the diameter D₁ as a prosthetic device/implant 12 is advanced through the sheath 8, without lengthening and/or without constricting. Thus, the force required to push the prosthetic implant 12 through the sheath 8 is significantly reduced.

Additionally, because of the radial force applied by the elastic third layer 106, the radial expansion of the sheath 8 can be localized to the specific portion of the sheath 8 occupied by the prosthetic device. For example, with reference to FIG. 14, as the prosthetic device/implant 12 moves distally through the sheath 8, the portion of the sheath 8 immediately proximal to the prosthetic device (for example, implant 12) can radially collapse back to the initial diameter D₁ under the influence of the elastic third layer 106. The inner layer 102 and the outer layer 108 can also buckle as the circumference of the sheath 8 is reduced, causing the ridges 126 and the valleys 128 to reform. This can reduce the size of the sheath 8 required to introduce a prosthetic device of a given size. Additionally, the temporary, localized nature of the expansion can reduce trauma to the blood vessel into which the sheath 8 is inserted, along with the surrounding tissue, because only the portion of the sheath 8 occupied by the prosthetic device expands beyond the sheath's natural diameter and the sheath 8 collapses back to the initial diameter once the device has passed. This limits the amount of tissue that must be stretched in order to introduce the prosthetic device, and the amount of time for which a given portion of the vessel must be dilated.

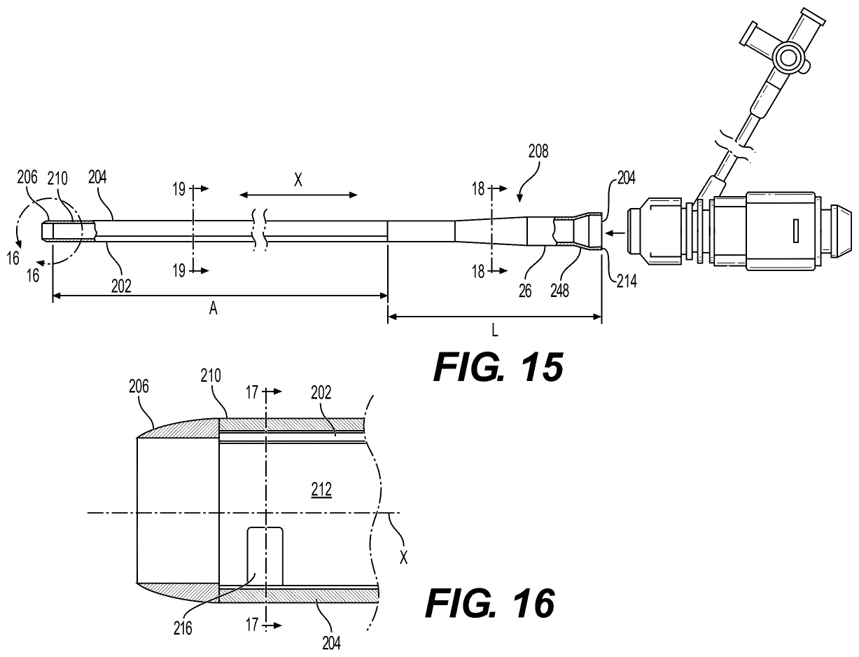

In another example layered sheath 208 structure, FIGS. 15-23 illustrate various features of the coaxial layered structure of the expandable sheath 8 of FIG. 1 according to another aspect. Similar reference numbers are used to describe like elements. It is to be understood that the variations (for example, materials and alternate configurations) described herein with reference to FIGS. 11-14 can also apply to the example shown in FIGS. 15-23. Furthermore, the variations described herein with reference to FIGS. 15-23 can also be applied to the sheath described in FIGS. 11-14.

Similar to various implementations of the sheath 8 described herein in reference to FIGS. 11-14, the sheath 208 of FIGS. 15-23 includes a plurality of layers. For example, the sheath 208 illustrated in FIGS. 15-23, also includes an inner layer 202 and an outer layer 204 disposed around the inner layer 202. The inner layer 202 can define a lumen 212 through which the delivery apparatus/implant 12 travels into the patient's vessel in order to deliver, remove, repair, and/or replace a prosthetic device, moving in a direction along the longitudinal axis X. Similar to the sheath 208 illustrated in FIGS. 11-14, as the prosthetic device passes through the sheath 208, the sheath 208 locally expands from a first, resting/unexpanded diameter to a second, expanded diameter to accommodate the prosthetic device. After the prosthetic device passes through a particular location of the sheath 208, each successive expanded portion or segment of the sheath 208 at least partially returns to the smaller, resting/unexpanded diameter. In this manner, the sheath 208 can be considered self-expanding, in that it does not require use of a balloon, dilator, and/or obturator to expand.

Similar to the examples herein, the inner layer 202 and outer layer 204 can comprise any suitable materials. Suitable materials for the inner layer 202 include polytetrafluoroethylene (PTFE), ethylene tetrafluoroethylene (ETFE), nylon, polyethylene, polyether block amide (for example, Pebax), and/or combinations thereof. In some implementations, the inner layer 202 can optionally include a lubricious, low-friction, or hydrophilic material, such as PTFE. Such low coefficient of friction materials can facilitate passage of the prosthetic device through the lumen defined by the inner layer 202. In some examples, the inner layer 202 can have a coefficient of friction of less than about 0.1. Some examples of the sheath 208 can optionally include a lubricious liner on the inner surface of the inner layer 202. Examples of suitable lubricious liners include materials that can further reduce the coefficient of friction of the inner layer 202, such as PTFE, polyethylene, polyvinylidene fluoride, and combinations thereof. Suitable materials for a lubricious liner also include other materials desirably having a coefficient of friction of about 0.1 or less.

Suitable materials for the outer layer 204 include nylon, polyethylene, Pebax, HDPE, polyurethanes (for example, Tecoflex), and other medical grade materials. In one implementation, the outer layer 204 can comprise high density polyethylene (HDPE) and Tecoflex (or other polyurethane material) extruded as a composite. In some implementations, the Tecoflex can act as an adhesive between the inner layer 202 and the outer layer 204 and may only be present along a portion of the inner surface of the outer layer 204. Other suitable materials for the inner layer 202 and outer layer 204 are also disclosed in U.S. Pat. Nos. 8,690,936 and 8,790,387, which are incorporated herein by reference.

Additionally, some examples of the sheath 208 include an optional exterior hydrophilic coating on the outer surface of the outer layer 204. Such a hydrophilic coating can facilitate insertion of the sheath 208 into a patient's vessel. Examples of suitable hydrophilic coatings include the Harmony™ Advanced Lubricity Coatings and other Advanced Hydrophilic Coatings available from SurModics, Inc., Eden Prairie, MN. DSM medical coatings (available from Koninklijke DSM N. V, Heerlen, the Netherlands), as well as other hydrophilic coatings (for example, PTFE, polyethylene, polyvinylidene fluoride), are also suitable for use with the sheath 8.

FIG. 16 provides a partial cross-section of the distal end of the sheath 208along section line 16-16 identified in FIG. 15. As described herein, the sheath 208 can be inserted into a vessel (for example, the femoral or iliac arteries) by passing through the skin of patient, such that a soft tip portion 206 at the distal end 210 of the sheath 208 is the first portion of the sheath 208 inserted into the vessel. As best seen in FIG. 16, the soft tip portion 206 can comprise, in some examples, low density polyethylene (LDPE) and can be configured to minimize trauma or damage to the patient's vessels as the sheath 208 is navigated through the vasculature. For example, in some implementations, the soft tip portion 206 can be slightly tapered to facilitate passage through the vessel. In some implementations, the soft tip portion 206 can be secured to the distal end 210 of the sheath 208, such as by thermally bonding the soft tip portion 206 to the inner layer 202 and outer layer 204 of the sheath 208. Such a soft tip portion 206 can be provided with a lower hardness than the other portions of the sheath 208. In some examples, the soft tip portion 206 can have a Shore hardness from about 25 D to about 40 D. The soft tip portion 206 is configured to be radially expandable to allow a prosthetic device to pass through the distal opening of the sheath 208. For example, in some implementations, the soft tip portion 206 can be formed with a weakened portion, such as an axially extending score line or perforated line that is configured to split and allow the soft tip portion 206 to expand radially when the prosthetic device passes therethrough.

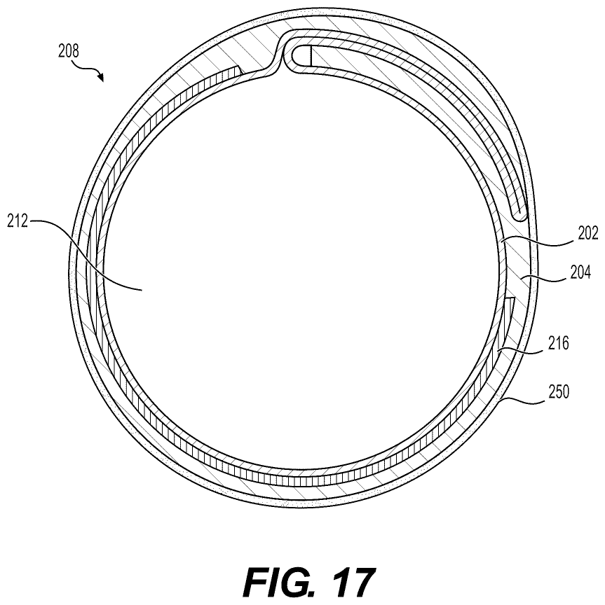

FIG. 17 shows a cross-section view of the sheath 208 taken near the distal end 210 of the sheath 208 as indicated by section line 17-17 in FIG. 16. As illustrated in FIGS. 16 and 17, the sheath 208 can optionally include at least one radiopaque filler or marker. In some implementations, the radiopaque filler or marker can include a discontinuous, or C-shaped, band (marker 216) positioned near the distal end 210 of the sheath 208. The marker 216 can be associated with the inner layer 202 and/or outer layer 204 of the sheath 208. For example, in some implementations, as shown in FIG. 17, the marker 216 can be positioned between the inner layer 202 and the outer layer 204. In other examples, the marker 216 can be associated with the outer surface of the outer layer 204. In some examples, the marker 216 can be embedded or blended within the inner layer 202 and/or outer layer 204.

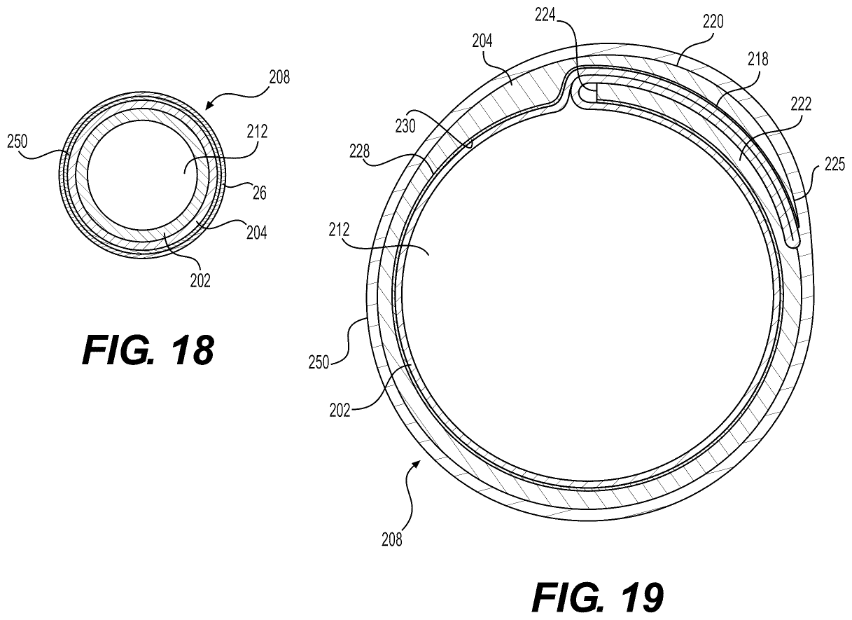

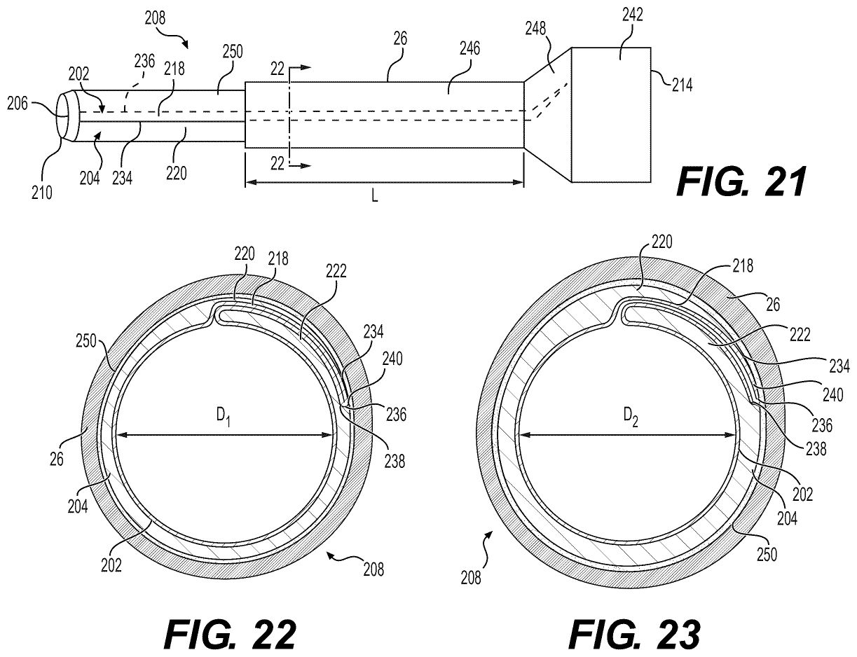

FIGS. 18 and 19 show additional cross sections taken at different points along the sheath 208. FIG. 18 shows a cross-section of a segment of the sheath 208 near the proximal end 214 of the sheath 208, as indicated by section line 18-18 in FIG. 15. At this location, the sheath 208 includes the inner layer 202, outer layer 204, elastic outer layer 250/outer jacket, and the strain relief layer 26. At this location, near the proximal end of the sheath 208, the inner layer 202 and outer layer 204 are substantially tubular. Here the inner layer 202 and outer layer 204 can be formed without any slits or folded portions in the layers. By contrast, as described herein, the inner layer 202 and outer layer 204 at different locations along the sheath 208, for example, at the point indicated by section line 19-19 in FIG. 15 and/or the point indicated by line section 22-22 in FIG. 21, can have a different configuration.

As shown in FIG. 19, the inner layer 202 can be arranged to form a substantially cylindrical lumen 212 therethrough. Inner layer 202 can include one or more folded portions 218. In the implementation shown in FIG. 19, inner layer 202 is arranged to have one folded portion 218 that can be positioned on either side of the inner layer 202. The inner layer 202 can be continuous, in that there are no breaks, slits, or perforations in inner layer 202. The outer layer 204 can be arranged in an overlapping fashion such that an overlapping portion 220 overlaps at least a part of the folded portion 218 of the inner layer 202. As shown in FIG. 19, the overlapping portion 220 also overlaps an underlying portion 222 of the outer layer 204. The underlying portion 222 can be positioned to underlie both the overlapping portion 220 of the outer layer 204, as well as the folded portion 218 of the inner layer 202. Thus, the outer layer 204 can be discontinuous, in that it includes a slit or a cut in order to form the overlapping portions 220 and underlying portions 222. In other words, a first edge 224 of the outer layer 204 is spaced apart from a second edge 225 of the outer layer 204 so as not to form a continuous layer.

As shown in FIG. 19, the sheath 208 can optionally include a thin layer of bonding or adhesive material 228 positioned between the inner layer 202 and outer layer 204. In some implementations, the adhesive material 228 can comprise a polyurethane material such as Tecoflex. The adhesive material 228 can be positioned on an inner surface of at least a portion of the outer layer 204 so as to provide adhesion between selected portions of the inner layer 202 and outer layer 204. For example, in some implementations, the outer layer 204 may only include a Tecoflex layer (adhesive material 228) around the portion of the inner surface 230 that faces the lumen-forming portion of the inner layer 202. In other words, the Tecoflex layer can be positioned so that it does not contact the folded portion 218 of the inner layer 202 in some implementations. In other implementations, the Tecoflex layer can be positioned in different configurations as desired for the particular application. For example, as shown in FIG. 19, the Tecoflex layer can be positioned along the entire inner surface 230 of the outer layer 204. In an alternative example, the Tecoflex layer can be applied to the outer surface of the inner layer 202 instead of the inner surface of the outer layer 204. The Tecoflex layer can be applied to all or selected portions on the inner layer 202; for example, the Tecoflex layer can be formed only on the portion of the inner layer 202 that faces the lumen-forming portion of the outer layer 204 and not on the folded portion 218. The configuration of FIG. 19 allows for radial expansion of the sheath 8 as an outwardly directed radial force is applied from within, for example, by passing a medical device such as a prosthetic heart valve through the lumen 212. As radial force is applied, the folded portion 218 can at least partially separate, straighten, and/or unfold, and/or the overlapping portion 220 and the underlying portion 222 of the outer layer 204 can slide circumferentially with respect to one another, thereby allowing the diameter of lumen 212 to enlarge.

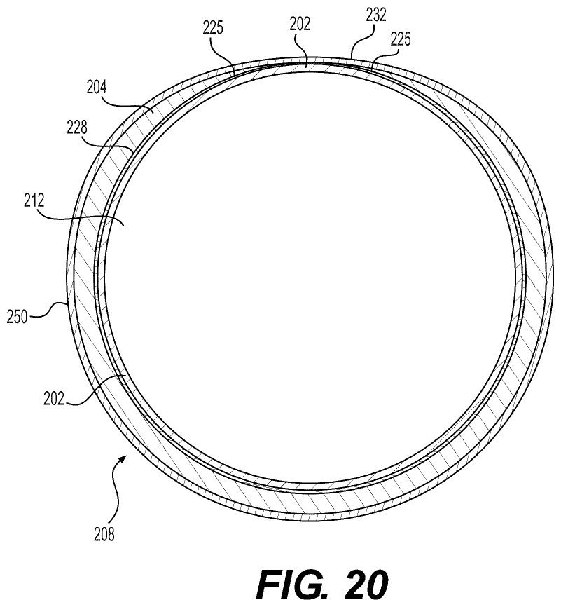

In this manner, the sheath 208 is configured to expand from a resting/unexpanded configuration (FIG. 19) to an expanded configuration shown in FIG. 20. In the expanded configuration, as shown in FIG. 20, an annular gap 232 can form between the longitudinal edges of the overlapping portion 220 and the underlying portion 222 of the outer layer 204. As the sheath 208 expands at a particular location, the overlapping portion 220 of the outer layer 204 can move circumferentially with respect to the underlying portion 222 as the folded portion 218 of the inner layer 202 unfolds. This movement can be facilitated by the use of a low-friction material for inner layer 202, such as PTFE. Further, the folded portion 218 can at least partially separate and/or unfold to accommodate a medical device having a diameter larger than that of lumen 212 in the resting/unexpanded configuration. As shown in FIG. 20, in some implementations, the folded portion of the inner layer 202 can completely unfold, so that the inner layer 202 forms a cylindrical tube at the location of the expanded configuration.

Similar to the example sheath 8 in FIG. 14, the sheath 208 is configured to locally expand at a particular location corresponding to the location of the medical device along the length of the lumen 212, and then locally contract once the medical device has passed that particular location. Thus, a bulge may be visible, traveling longitudinally along the length of the sheath 208 as a medical device is introduced through the sheath 208, representing continuous local expansion and contraction as the device travels the length of the sheath 208. Each segment of the sheath 208 will locally contract after removal of any radial outward force such that the sheath 208 at least partially returns to the original resting/unexpanded diameter of lumen 212. Similar to the example sheath 8 described herein, an elastic outer layer 250 can (optionally) be provided along the sheath 208, urging the inner layer 202 and outer layer 204 back towards the unexpanded configuration.

The layers 202, 204 of sheath 208 can be configured having the folded portion 218 as shown in FIG. 19 along at least a portion of the length of the sheath 208. In some examples, the inner layer 202 and outer layer 204 can be configured as shown in FIG. 19 along the length A (FIG. 15) such that the folded portion 218 extends from a location adjacent the soft tip portion 206 to a location closer to the proximal end 214 of the sheath 208, adjacent and/or under the distal end of the strain relief layer 26. In this matter, the sheath 208 is expandable and contractable only along a portion of the length of the sheath 208 corresponding to length A. In some implementations, this portion of the sheath 208 corresponds to the section of the sheath 208 inserted into the narrowest section of the patient's vasculature.

In some examples, the folded portion 218 extends from a location adjacent the soft tip portion 206 under the strain relief layer 26, as illustrated in FIG. 21. In this example, the folded structure of the inner layer 202 extends from the soft tip portion 206, under the strain relief layer 26 and along the tapered portion 248 of the strain relief layer 26.

FIGS. 22 and 23 provide cross-section views of the sheath 208 taken along the strain relief layer 26 at section line 22-22 in FIG. 21. In this example, the folded portion 218 of the inner layer 202 extends under the strain relief layer 26. FIG. 22 shows a cross-section of the sheath 208 in a resting/unexpanded configuration having an inner diameter D₁. FIG. 23 shows a cross-section of the sheath 208 in a (partially) expanded configuration, having an inner diameter D₂, where D₂ is greater than D₁.

As shown in FIGS. 22 and 23, in some examples, the overlapping portion 220 does not overlap the entire folded portion 218 of the inner layer 202, and thus a portion of the folded portion 218 can be located directly adjacent/aligned to the strain relief layer 26 in locations where the strain relief layer 26 is present. In locations where the strain relief layer 26 is not present, part of the folded portion 218 may be visible from the outside of the sheath 208, as seen in FIG. 21 (and/or visible through an elastic outer layer 250 described in more detail herein). In some implementations, the sheath 208 can include a longitudinal seam 234 where the overlapping portion 220 terminates at the folded portion 218. In use, the sheath 208 can be positioned such that the seam 234 is posterior to the point of the sheath 208 that is 180 degrees from the seam 234 (for example, facing downward in the view of FIG. 21). As shown in FIG. 21, in some implementations, the seam 234 need not extend the entire length of the sheath 208, and ends at a transition point between portions of the sheath 208 having a folded inner layer and portions of the sheath 208 not having a folded inner layer.

In some examples, the folded portion 218 can include a weakened portion 236. In some implementations, the weakened portion 236 includes a longitudinal perforation, score line, and/or slit, along at least a portion of the length of the inner layer 202. The weakened portion 236/slit allows for the two adjacent ends 238, 240 of the folded portion 218/inner layer 202 to move relative to one another as the sheath 208 expands/moves to the expanded configuration shown in FIG. 23. For example, in some implementations, the sheath 208 locally expands as a medical device is inserted therethrough, causing the weakened portion 236 to split/separate.

In each of the example sheaths described herein (for example sheath 8 and sheath 208), the sheath 208 may include an elastic outer layer 250 that expands with the sheath 208. While the elastic outer layer 250 is described in reference to sheath 208 provided in FIGS. 15-23, it is contemplated that the elastic outer layer 250 may also be provided on sheath 8 provided in FIGS. 11-14, and any other sheath described herein. The elastic outer layer 250 can provide an inwardly directed radial force that directs the sheath 208 towards a folded/unexpanded configuration. Similar to the strain relief layer 26, elastic outer layer 250 can also provide hemostasis helping to prevent blood loss during implantation of the prosthetic device and/or placement of the sheath 208 within the blood vessel.

The elastic outer layer 250 can be positioned around at least a portion of the strain relief layer 26, outer layer 204 and/or the inner layer 202 of the sheath 208 (and/or outer layer 108, inner layer 102). As illustrated in FIGS. 21-23, the outer layer 250 can surround the entire circumference of outer layer 204, and can extend longitudinally along any portion of the length of the sheath 208, including along (over and/or under) the strain relief layer 26. The elastic outer layer 250 extends for a length along at least a portion of the main body of the sheath 208. In some examples, the elastic outer layer 250 extends to a point adjacent the distal end 210 of the sheath 208. In some implementations, the elastic outer layer 250 or can extend all the way to the distal end 210 of sheath 208. In some implementations, the elastic outer layer 250 extends over the entire length of the sheath 208.

As shown in FIGS. 17-20, 22 and 23, the elastic outer layer 250 can be a continuous tubular layer, without slits or other discontinuities. The elastic outer layer 250 extends between strain relief layer 26 and the outer surface of the outer layer 204. In other examples, the elastic outer layer 250 extends over the outer surface of the strain relief layer 26 and the outer surface of the outer layer 204. In some examples, the elastic outer layer 250 extends both over the strain relief layer 26 and/or between the outer layer 204 of the sheath 208 and the strain relief layer 26.

The elastic outer layer 250 can comprise any pliable, elastic material(s) that expands and contracts, preferably with a high expansion ratio. Preferably, the materials used can include low durometer polymers with high elasticity, such as Pebax, polyurethane, silicone, and/or polyisoprene. Materials for the elastic outer layer 250 can be selected such that it does not impede expansion of the inner layer 202 and outer layer 204 of the sheath 208. The elastic outer layer 250 can have a thickness ranging from, for example, about 0.001′ to about 0.010.′ In some implementations, the elastic outer layer 250 can have a thickness ranging from about 0.003′ to about 0.006.′ The elastic outer layer 250 can be configured to stretch and expand as the sheath 208 expands, as shown in the expanded configuration in FIG. 20.

As illustrated in FIGS. 2, 15 and 21, the sheath 8 and sheath 208 includes a strain relief layer 26. While the strain relief layer 26 is described in reference to sheath 208 provided in FIGS. 15-23, it is contemplated that the strain relief layer 26 may also be included with sheath 8 provided in FIGS. 11-14, and any other sheath described herein.

The strain relief layer 26/tube is provided adjacent the proximal end of the sheath 208 and extends along/over the outer surface of the sheath 208. In some examples, the strain relief layer 26 is provided over the outer layer 204 (and/or outer layer 208) of the sheath 208. The strain relief layer 26 forms a smooth transition between the sheath hub 20 and the sheath 208 and facilitates mating of the sheath 208 with the sheath hub 20.

Additionally, and as will be described in more detail herein, the strain relief layer 26 provides a region of higher durometer or stiffness that restricts expansion of the underlying sheath layers. This helps to ensure hemostasis between the portions of the sheath 8, 208 inside the patient and the sheath hub (external to the patient). The increased durometer and/or stiffness along the strain relief layer 26 prevents blood from flowing between the various layers of the sheath 208 exterior to the patient during the procedure, helping to withstand the blood pressure that would otherwise cause the sheath 208 to “balloon up” with body fluid/blood. Additionally, in some implementations, the strain relief layer 26 can be sized and configured to form a seal with the patient's artery when inserted, such that blood is substantially prevented from flowing between the strain relief layer 26 and the vessel wall. For example, although the strain relief layer 26 does not extend all the way to the distal end 210 of the sheath 208, the strain relief layer 26 can extend distally enough along the sheath 208 that when the sheath 208 is inserted into the patient's blood vessel, a portion of the strain relief layer 26 extends through and seals against the arteriotomy site.

As described herein, the strain relief layer 26 is provided over the outer layer 204 of the sheath 208 (and/or outer layer 108). In some implementations, the strain relief layer 26 can be bonded to the outer layer 204 to prevent the strain relief layer 26 from sliding over the outer layer 204 and “bunching up” in response to the friction forces applied by the surrounding tissue during insertion of the sheath 208 into the patient's vasculature. For example, the strain relief layer 26 can be bonded at the proximal end and/or distal end of the outer layer 204. In some implementations, at the proximal and distal ends, the strain relief layer 26 can be bonded to the outer layer 204 around the full circumference of the outer layer 204. In some implementations, at the distal end of the sheath 208, the strain relief layer 26 can alternatively be bonded to the inner layer(s) of the sheath 208. For example, the strain relief layer 26 can be bonded to the distal end surface of the inner layer 202.

FIGS. 18, 22 and 23 illustrate cross-section views of the sheath 208 along the strain relief layer 26. FIG. 18 shows a cross-section of a segment of the sheath 208 near the proximal end 214 of the sheath 208, as indicated by line 18-18 in FIG. 15. Similarly, FIGS. 22 and 23 show cross-section segments of various example sheaths near the proximal end 214 of the sheath 208 and closer to the distal end of the strain relief layer 26, as indicated by section line 22-22 in FIG. 21. As illustrated in each of FIGS. 15-23, the sheath 208 at this location can comprise an inner layer (liner) 202, outer layer 204, adhesive material 228 layer, an optional elastic outer layer 250, and the strain relief layer 26.

The strain relief layer 26 extends circumferentially around at least a portion of the inner layer 202 and outer layer 204. The strain relief layer 26 extends from the proximal end 214 of the sheath 208 in a direction towards the distal end 210 of the sheath 208. As shown in FIG. 21 (and FIG. 15), the strain relief layer 26 extends for a length L along at least a portion of the main body of the sheath 208. In some examples, the strain relief layer 26 extends to a point adjacent the distal end 210. In further examples, the strain relief layer 26 extends all the way to the distal end 210 of sheath 208. In some examples, the longitudinal length L of the strain relief layer 26 can range from about 10 cm to the entire length of the sheath 208.

In some implementations, the strain relief layer 26 extends to/adjacent the proximal end 214 of the sheath 208 and provides a compression fit over the distal end of the sheath hub 20 thereby coupling the sheath 208 to the sheath hub 20. Additionally, or alternatively, the strain relief layer 26 can be secured between the sheath hub 20 and the sheath hub cap 22 (or other fastening device for coupling the proximal end of the sheath 208 to the sheath hub 20), as illustrated in FIGS. 5A and 5B. In some examples, the strain relief layer 26 does not extend all the way to the proximal end 214 of the sheath 208.

It is understood that strain relief layer 26, as shown herein, can have similar composition and characteristics of the inner layer 202 and outer layer 204 as disclosed herein. Various compositions are disclosed, for example, in Application No. PCT/US2021/301275, entitled “Expandable sheath for introducing an endovascular delivery device into a body,” the disclosure of which is herein incorporated by reference.

In some implementations, the strain relief layer 26 can comprise a lubricious, low-friction, and/or relatively non-elastic material. Preferably the materials used can include high durometer polymers, with low elasticity. In some examples, the strain relief layer 26 is composed of the same and/or similar material to the inner layer 202 and/or outer layer 204 (including inner layer 102 and/or outer layer 108). For example, as described herein regarding the inner layer 102 and outer layer 108, exemplary materials can include polyurethane (for example, high density polyethylene), ultra-high-molecular-weight polyethylene (UHMWPE) (for example, Dyneema®), high-molecular-weight polyethylene (HMWPE), or polyether ether ketone (PEEK). Other suitable materials for the strain relief layer 26 can include polyimide, polytetrafluoroethylene (PTFE), expanded polytetrafluoroethylene (ePTFE), ethylene tetrafluoroethylene (ETFE), nylon, polyethylene, polyamide, polyether block amide (for example, Pebax), and/or combinations of any of the above. Materials for the strain relief layer 26 can be selected such that it the material properties help to impede expansion of the underlying layers of the sheath 208.

The strain relief layer 26 can have a thickness ranging from, for example, about 0.001′ to about 0.010.′ In some implementations, the strain relief layer 26 can have a thickness of from about 0.003′ to about 0.006.′ The wall thickness is measured radially between the inner surface of the strain relief layer 26 and the outer surface of the strain relief layer 26.

In alternative examples, the material composition and/or wall thickness can change along the length of the strain relief layer 26. For example, the strain relief layer 26 can be provided with one or more segments, where the composition and/or thickness changes from segment to segment. In an example aspect, the Durometer rating of the composition changes along the length of the strain relief layer 26 such that segments near the proximal end comprise a stiffer material or combination of materials, while segments near the distal end comprise a softer material or combination of materials. Similarly, the wall thickness of the strain relief layer 26 in segments near the proximal end can be thicker/greater than the wall thickness of the elastic outer layer 250 near the distal end.

As illustrated in FIGS. 15, 21 and 24, the strain relief layer 26 has a proximal end and a distal end and a central lumen extending longitudinally therethrough. The strain relief layer 26 includes a generally tubular shaped proximal portion 242 adjacent the proximal end of the strain relief layer 26, and a generally tubular shaped distal portion 246 adjacent the distal end of the strain relief layer 26. In some implementations, the strain relief layer 26 includes a frustoconical shaped tapered portion 248 extending between the proximal portion 242 and the distal portion 246 of the strain relief layer 26, such that the diameter of the strain relief layer 26 at the proximal portion 242 is greater than the diameter of the strain relief layer 26 at the distal portion 246 of the strain relief layer 26. The tapered portion 248 and the flared proximal portion 242 help ease the transition of the medical device/delivery system when passing between the larger diameter sheath hub 20 to the smaller diameter of the sheath 208. As described herein, in some implementations, the tapered portion 248 and/or flared portion 242 provide a radially inward compressive force against the medical device/delivery system as it is advanced through the sheath hub 20 and into the sheath 208, thereby helping to compress the medical device/delivery system to a smaller diameter. As a result, the push forces required to advance the medical device/delivery system into the sheath 208 are reduced, and reducing the likelihood of damage to the sheath 208 and/or medical device/delivery system.

As described herein, the strain relief layer 26 is made of a material that is stiffer than the other sheath 208 layers such that the strain relief layer 26 inhibits expansion of the portion of the sheath disposed along/under the strain relief layer 26. Because radial expansion is limited along the strain relief layer 26, higher push forces are necessary to advance the medical device (implant 12) through the central lumen of the portion of the sheath 208 corresponding to the strain relief layer 26. In some examples, the highest push force through the sheath 208 are experienced near the ends (for example, proximal and distal ends) of the strain relief layer 26.

In some implementations, the thickness and/or composition of the strain relief layer 26 can be adjusted to improve the performance of the strain relief layer 26 and to reduce the push force. While the steps and benefits of predilating the sheath are described in reference to sheath 208, it is contemplated corresponding steps and benefits may also be provided with respect the sheath 8, and any other sheath described herein. As described herein, pre-dilating the sheath 208, or a portion thereof, can help to reduce push forces required to insert the medical device/delivery system through the central lumen of the sheath 208.

Pre-dilating the sheath 208 releases and/or loosens any bonding or adhesion of the sheath 208 layers that occurs during the manufacturing process, for example, bonding between the inner layer 102, 202 and outer layer 108, 204, bonding between the adjacent ridges 126, bonding between the folded portion 218 and outer layer 204, bonding between the inner layer 102, 202 and the outer layer 108, 204 and the strain relief layer 26, bonding or resistance to expansion along the strain relief layer 26 or at the distal tip. In some implementations, pre-dilating the strain relief layer 26 can also break or separate the weakened portion 236 of folded portion 218 of the inner layer 202, separating adjacent ends 238, 240 of the folded portion 218, as described herein and illustrated in FIG. 23. With the 208 layers able to move freely with respect to the other, the medical device/delivery system is pushed through the sheath 208 lumen at a much lower force.

In some instances, the sheath 208 is pre-dilated by passing a relatively large dilator (for example, 22 French dilator) through the sheath 208. Pre-dilating can include passing a dilator through the strain relief layer 26 and/or distal tip of the sheath 208. The pre-dilating step can be done during manufacturing and/or during sheath 208 preparation, prior to sheath 208 insertion into the patient and/or with the sheath 208 at least partially inserted into the patient. However, in some instances, pre-dilating the sheath 208 can result in a partially open seam or edge between the now-released layers of the sheath 208, may introduce potential for vascular complications, for example, difficulty or vessel injury during insertion, movement and/or withdrawal of the pre-dilated sheath 208, and it may also be aesthetically unacceptable to physicians.

The devices, systems, and methods described herein provide for systems and methods of pre-expanding the sheath 208 while also preventing the longitudinal seam or open edge (for example, seam 234 described herein in reference to FIG. 21) of the expanded sheath 208 layers from propagating axially or from further opening radially after pre-expansion during, for example, sterilization or aging of the sheath 208. The system described herein allows the sheath 208 to be dilated during manufacturing, not requiring the medical staff to dilate during sheath prep, reducing preparation time and eliminating unnecessary opportunities for error and/or inadvertent damage to the sheath 208.

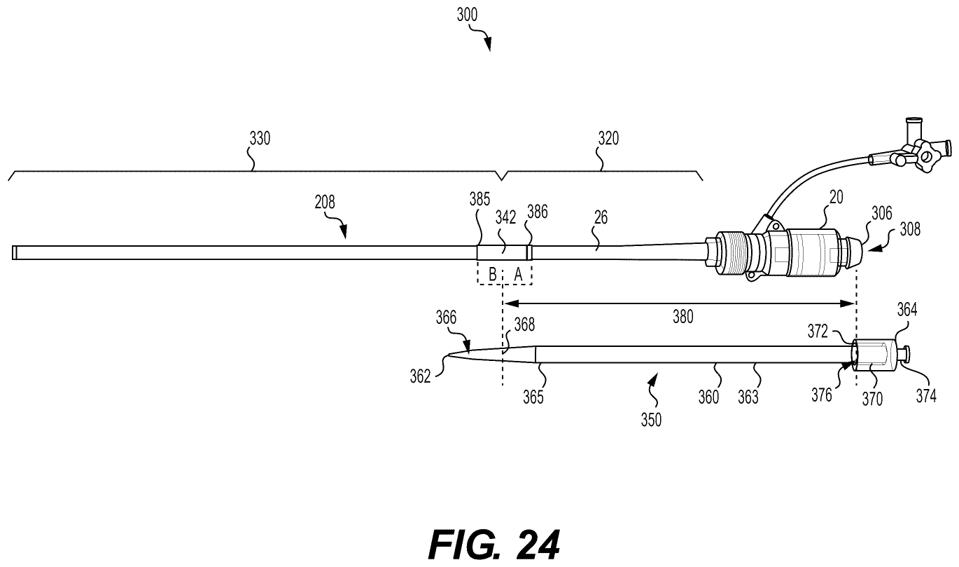

FIG. 24 shows an example system 300 including a sheath 208 and dilator 350. While the system 300 and corresponding method of pre-dilating the sheath is described in reference to sheath 208 provided in FIGS. 15-23, it is contemplated that system 300 and corresponding method may also be used with sheath 8, provided in reference to FIGS. 11-14, or any other expandable sheath described herein.

At least a portion of the sheath 208 (for example, inner layer 102, 202 and/or strain relief layer 26) is configured to locally expand from an unexpanded configuration (for example, FIG. 18-19), in which the lumen 212 has a first diameter to an expanded configuration (for example, FIG. 20) in which the lumen 212 has a second, larger, diameter. Expansion of the sheath 208 is directed in response to an outwardly directed radial force exerted on the lumen 212 by the expansion element 365 of the dilator 350 (and/or medical device) received within the sheath 208. The sheath 208 then locally contracts at least partially back toward the unexpanded configuration as the dilator 350 (and/or medical device) passes through the lumen 212. As will be described in more detail herein, the restraining member 385 limits expansion of the sheath 208 (for example, inner layer 202 and/or strain relief layer 26) proximate the restraining member 385. By limiting expansion/unfolding of the sheath 208, use of the restraining member 385 prevents the longitudinal seam 234 or an open edge between adjacent layers of the dilated sheath 208 from propagating axially and/or from further opening radially after pre-expansion.

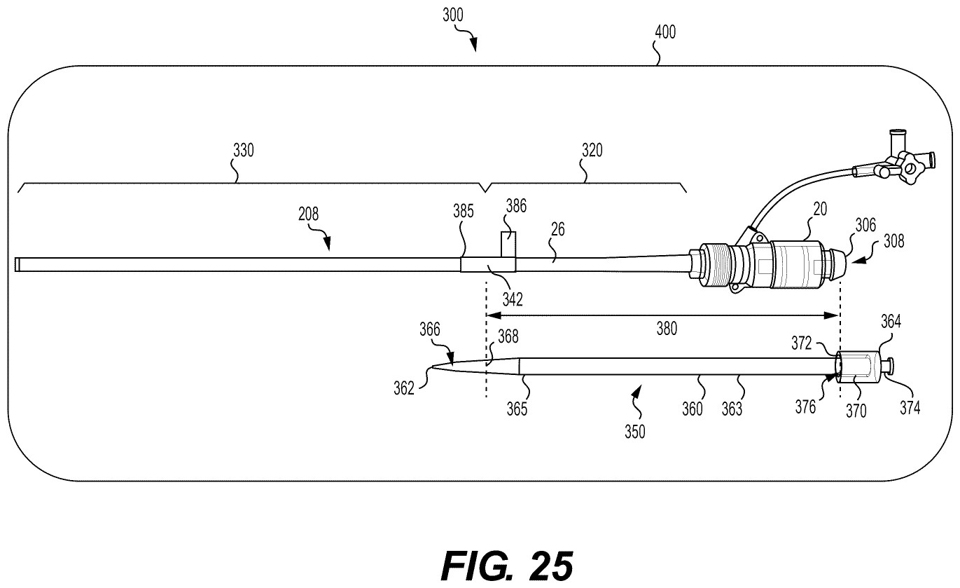

The representative sheath illustrated in FIG. 24 (and FIG. 25) corresponds to expandable sheath configurations described herein, for example, the layered configuration illustrated in FIGS. 11-14 and/or FIGS. 15-23. The sheath 208 includes a continuous inner layer (for example, inner layer 202) defining a central lumen extending therethrough and corresponding outer layers (for example, outer layer 204 In the following description, the sheath 208 will be described in reference to the layered structure of the sheath 208 of FIGS. 15-23. However, it is contemplated that the layered sheath 8 structure of FIGS. 11-14 could be used. For example, in some implementations, the sheath 208 includes a continuous inner layer 202 defining a lumen 212 therethrough. The inner layer 202 includes a proximal portion 320 and a main body portion 330 and a folded portion 218 extending along a length of the inner layer 202. In some implementations, the sheath 208 includes an outer layer (for example, outer layer 204) provided over the inner layer 202, and under or over the tubular strain relief layer 26. The sheath 208 includes a tubular strain relief layer 26 provided over the inner layer 202 at a proximal end of the sheath 208 and extending along at least a portion of the length of the sheath 208. As described herein, the strain relief layer 26 provides a region of higher durometer or stiffness that restricts expansion of the underlying sheath layers. The strain relief layer 26 is provided at the proximal end of the sheath 208 and extends along at least a portion of the length of the sheath 208. Similar to the inner layer 202 and outer layer 204, at least a portion of the strain relief layer 26 is configured to locally expand from an unexpanded configuration at a first diameter to an expanded configuration at a second, larger, diameter due to an outwardly directed radial force exerted on the lumen 212 of the inner layer 202 by the dilator 350 (or a medical device) against the inner layer 202, and then locally contract at least partially back to the unexpanded configuration as the dilator 350 (or medical device) passes through the lumen 212.

As illustrated in FIG. 24, the sheath 208 includes a restraining member 385 provided over the sheath 208 for limiting expansion/unfolding of the adjacent/underlying portion of the sheath 208. In some implementations, the restraining member 385 limits expansion/unfolding of the folded portion 218 of the inner layer 202 and/or movement between the inner layer 202 and outer layer 204 when the sheath moves from the unexpanded to the expanded configuration. As a result, this prevents the longitudinal seam 234 from forming/opening between the overlapping portion 220 of the outer layer 202 and the folded portion 218 of the inner layer 202 (FIG. 21). This also helps to prevent an opening/gap from forming between adjacent layers of the folded portion 218 from propagating axially (for example, proximally or distally along the length of the sheath 8). And may also help prevent any opening/gap formed between the adjacent layers of the folded portion 218 from further opening radially after pre-expansion.

In some implementations, as illustrated in FIG. 24, the restraining member 385 is positioned over the distal end 342 of the strain relief layer 26. As a result, the restraining member 385 limits expansion of the underlying and/or adjacent portion of at least one of the inner layer 202 and strain relief layer 26. For example, in some implementations, the restraining member 385 is provided over a length of sheath 208 at a location corresponding to the distal end 342 of the strain relief layer 26 and extends along a length (portion A) of the strain relief layer 26 extending from the distal end 342 toward a proximal end of the strain relief layer 26. As provided in FIG. 24, in some implementations the restraining member 385 extends along a second length (portion B) of the sheath 208 extending from the distal end 342 of the strain relief layer 26 toward a distal end of the sheath 208.

The restraining member 385 can be constructed from a material having lower elasticity than the underlying layers of the sheath 208 and/or strain relief layer 26 so that the restraining member 385 limits and/or prevents radial expansion of the sheath 208/strain relief layer 26. In some implementations, the restraining member 385 is constructed from a tape, a shrink tube, an elastic tube, a packaging feature, or other structure or material provided over the sheath 208 for limiting radial expansion. In some implementations, the restraining member 385 is coupled to the sheath 208. For example, in some implementations, the restraining member 385 is coupled to the outer surface of the inner layer 202 and/or strain relief layer 26. In some implementations, the restraining member 385 is releasably coupled to the sheath 208. For example, some implementations, an inner surface of the restraining member 385 can include an adhesive (for example, temporary/releasable adhesive and/or resealable adhesive) for coupling the restraining member 385 to the sheath 208. In some implementations, the restraining member 385 includes a shrink tubing coupled to at least one of the inner layer 202 or the strain relief layer 26 by a shrink process (for example, a shrink heating process). In some implementations, the restraining member 385 includes a release feature 386 for removing the restraining member 385 from the sheath 208. For example, the release feature 386 can be used to partially or completely remove the restraining member 385 from the inner layer 202 and/or the strain relief layer 26. In some implementations, the release feature 386 can include at least one of a weakened portion (for example, perforation, score line, slit, etc.), or a pull tab, and/or line integral with the restraining member 385 that when activated, the restraining member 385 separates, at least partially, from or along the sheath 208.

In some implementations, as illustrated in FIG. 25, the release feature 386 is incorporated into packaging sized and configured to receive the sheath 208. FIG. 25 shows the sheath system 300 included in its corresponding packaging tray 400. The tray 400 includes indentations/recesses sized and configured to securely receive the components of the sheath system 300. When the release feature 386 is incorporated into the tray 400, removing the sheath 208 from the tray 400 also removes the restraining member 385 from the inner layer 202 and/or the strain relief layer 26. For example, in some implementations, a portion of the restraining member 385 and/or the release feature 386 (for example, pull tab or line) is fixedly coupled to the tray 400 such that when the sheath 208 is removed from the tray 400 the restraining member 385 is, at least partially, separated from and/or along the sheath 208. In some implementations, when separated from the sheath 208, at least a portion of the restraining member 385 may remain coupled to the tray 400. In some implementations, when separated from the sheath 208 the restraining member 385 can be separated from both the sheath 208 and the tray 400. In some implementations, as the sheath 208 is removed from the tray 400, the release feature 386 is activated and the restraining member 385 separated from and/or along the sheath 208. For example, in some implementations, the release feature 386 includes a weakened portion along the restraining member 385. As the sheath 208 is removed from the tray 400, separating or tearing along the weakened portion is triggered and the restraining member 385 is removed from or separated along the sheath 208.

The system 300 includes a dilator 350 sized and configured to be received within the central lumen 212 of the inner layer 202. As illustrated in FIG. 24, the dilator 350 includes an elongated dilator shaft 360 with an expansion element 365 provided thereon. As described herein, the sheath 208 locally expands from the unexpanded configuration to the expanded configuration in response to an outwardly directed radial force exerted on the central lumen 212 by the expansion element 365 of the dilator 350 (and/or a medical device).

In some implementations, the dilator shaft 360 includes a body portion 363 adjacent the proximal end 364 of the dilator shaft 360, and a tapered portion 366 extending from the distal end 362 of the dilator shaft 360 toward the body portion 363. In some implementations, the length of the dilator shaft 360 received within the hub opening 376 is adjustable to vary the dilator 350 length 380, i.e., the length of the dilator 350 extending from the dilator hub 370. The proximal end 364 of the dilator shaft 360 is coupled to a dilator hub 370 at the hub opening 376. In some implementations, the hub opening 376 extends from the hub distal end 372 toward the hub proximal end 374, and the dilator shaft 360 is fixedly coupled within the hub opening 376. In use, the dilator shaft 360 is inserted from the sheath hub proximal end 306, through the central lumen 308 of the sheath hub 20, and into the central lumen 212 of the sheath 208. In some implementations, the dilator hub 370 is coupled to the proximal end 306 of the sheath hub 20.

In some implementations, the expansion element 365 is provided on the body portion 363 of the dilator 350. In some implementations, as illustrated in FIG. 24, the expansion element 365 is defined by the body portion 363 of the dilator shaft 360. In other implementations, the expansion element 365 includes a projection extending radially from the outer surface of the dilator shaft 360. For example, the expansion element 365 can include a regular or irregular shaped projection extending from the outer surface of the dilator shaft 360 and around all or a portion of the circumference of the dilator 350.

In some implementations, the diameter of the expansion element 365 is greater than the unexpanded diameter of the sheath 208, such that movement of the expansion element 365 through the lumen 212 of the sheath 208 causes the sheath 208 to radially expand to a diameter larger than its unexpanded diameter. In some implementations, the diameter of the expansion element 365 is 22F. For example, in some implementations, the expansion element 365 has a diameter ranging from 12F to 24F. In another example, the expansion element 365 has a diameter ranging from 14F to 24F. In some examples, the expansion element 365 has a diameter ranging from 14F to 22F. The diameter of the expansion element 365 can be selected based on the unexpanded sheath 208 diameter and/or the delivery system/medical device diameter and the corresponding desired amount of the pre-expansion of the sheath 208.

A method of producing and/or manufacturing a pre-dilated expandable sheath for delivering a medical device is described herein. The method includes providing a radially expandable sheath according to any of the examples described herein. While the method of producing a pre-dilated sheath is described in reference to sheath 208 provided in FIGS. 15-23, it is contemplated that the method may also be used with sheath 8, provided in reference to FIGS. 11-14, or any other expandable sheath described herein. In some implementations, the sheath 208 includes a continuous inner layer 202 defining the lumen 212 of the sheath 8. The inner layer 202 includes a proximal portion 320 and a main body portion 330. In some implementations, the inner layer 202 includes a folded portion 218 extending along a length of the sheath 208 corresponding to the proximal portion 320 and/or main body portion 330. In some implementations, the sheath 208 includes a tubular strain relief layer 26 provided over the proximal portion 320 of the inner layer 202.

The method includes positioning a restraining member 385 over a portion of the sheath 208. In some implementations, the restraining member 385 is provided over, at least, the distal end 342 of the strain relief layer 26. The restraining member 385 provides an inwardly directed radial force that limits expansion of the adjacent portions of the inner layer 202 and/or strain relief layer 26, thereby preventing the longitudinal seam 234 and/or other open edge formed between adjacent layers of the dilated sheath 208 from propagating axially or further opening radially after pre-expansion. For example, the inwardly directed radial force provided by the restraining member 385 limits the unfolding of the folded portion 218 of the inner layer 202 proximate the restraining member 385 when the sheath 208 moves from the unexpanded configuration to the expanded configuration during advancement of the dilator 350.

By providing the restraining member 385 over the distal end 342 of the strain relief layer 26, the restraining member 385 is provided over the portion of the sheath 208 including the portion including the beginning of the exposed portion of the seam 234. As illustrated in FIG. 21, a proximal portion of seam 234 extending under the strain relief layer 26, and a distal portion of the seam 234 extending along the sheath 208 beyond the strain relief layer 26. In some implementations, as illustrated in FIG. 24, the restraining member 385 is provided over the sheath 208 at the distal end 342 of the strain relief layer 26, and also extends distally over a first length (portion A) of the sheath 208 toward the proximal end of the strain relief layer 26, and also along a second length (portion B) toward a distal end 210 of the sheath 208.

In some implementations, the restraining member 385 is releasably coupled to the sheath 208. For example, the restraining member 385 can be releasably coupled to the sheath 8 using a temporary adhesive applied to the inner surface of the restraining member 385 or corresponding portion of the sheath 208, for example, a corresponding portion of the outer surface of the inner layer 202 and/or the strain relief layer 26. In some implementations, the restraining member 385 comprises an adhesive tape that is applied to the outer surface of the sheath 208. In some examples, the restraining member 385 comprises a shrink tubing that is provided over the sheath 208 and a shrink process is applied, for example, a shrink heating process.

The method further includes introducing the dilator 350 into the proximal end 214 of the central lumen 212 of the 208 and advancing the dilator 350 through a desired length of the sheath 208. In some implementations, the dilator 350 is advanced through the proximal portion 320 of the inner layer 202 corresponding to the strain relief layer 26. As a result, the expansion element 365 exerts an outwardly directed radial force against the central lumen 212 of the sheath 208 and causes the inner layer 202 and the strain relief layer 26 proximate the expansion element 365 to locally expand from an unexpanded configuration to an expanded configuration. The inwardly directed radial force exerted by the restraining member 385 limits expansion and/or unfolding of the underlying portion of the sheath 208 as the dilator 350 moves through the central lumen 212, i.e., the restraining member 385 limits expansion/unfolding of the inner layer 202, outer layer 204, and/or strain relief layer 26 to prevent propagating unnecessary opening of the longitudinal seam 234 or other opening.

In some implementations, the dilator 350 is advanced through the proximal portion 320 of the inner layer 202 such that the expansion element 365 of the dilator 350 aligns with the distal end 342 of the strain relief layer 26, such that the distal end 342 of the strain relief layer 26 is expanded. For example, the dilator 350 can be advanced within the lumen 212 of the sheath 208 until the proximal end 368 of the tapered portion 366 of the dilator 350 and/or a distal end of the body portion 363 of the dilator shaft 360 aligns with the distal end 342 of the strain relief layer 26.

In some implementations, the dilator 350 is advanced through the proximal portion 320 of the inner layer 202 such that the expansion element 365 of the dilator 350 is located beyond the distal end 342 of the strain relief layer 26. As a result, the distal end 342 of the strain relief layer 26 and a portion of the main body portion 330 of the inner layer 202 are at least partially expanded. For example, the dilator 350 can be advanced within the lumen 212 of the sheath 208 until the proximal end 368 of the tapered portion 366 and/or a distal end of the body portion 363 of a dilator shaft 360 is located beyond the distal end 342 of the strain relief layer 26. In some examples, the expansion element 365 is used to expand/dilate a length of the main body portion 330 of the sheath extending 10-15 mm beyond the distal end 342 of the strain relief layer 26. In this example, expanding the length of the main body portion 330 of the sheath 208 beyond the distal end 342 of the strain relief layer 26 causes a corresponding length of the folded portion 218 to at least partially unfold as the bonding between the folded layers of the inner layer 202 is released. Providing the restraining member 385 over this portion of the sheath 208, limits the unfolding and any undesirable separating along the seam 234 or other opening between adjacent layers of the dilated sheath 208.

In some implementations, the method further includes heating the sheath 208. In some implementations, heating the sheath 208 in a partially expanded configuration heat sets the size (for example, width and/or length) of the seam 234 or open edge between adjacent layers of the dilated sheath 208. For example, the heating step can be provided by a heated sterilizing process and/or heat setting process applied to the sheath 208 in the expanded/dilated configuration. However, during the heating step, the sheath 208 is not heated at a temperature or for a duration sufficient to bond layers of the folded portion 218. In some implementations the heating step includes heating the sheath 208 at a temperature and for a duration corresponding to a sterilization process. For example, in some implementations, the sheath 208 is heated at a temperature of 60° C. In some implementations, the sheath 208 is heated for a duration greater than 12 hours. For example, the sheath 208 is heated at 60° C. for 24 hours. In some examples, the sheath 208 is heated at 60° C. for 26 hours.

In some implementations, the dilator 350 is coupled to the sheath hub 20 and/or the dilator hub 370 before the heating step. The dilator 350 can be advanced through the proximal portion 320 of the sheath 208 until the dilator hub 370 abuts the sheath hub 20. In some implementations, the dilator hub 370 is then coupled to the sheath hub 20 fixing the position of the dilator 350 within the lumen 212 of the sheath 208. In some examples, the dilator hub 370 is releasably coupled to the sheath hub 20 by a mechanical coupling including, for example, a press fit, interference fit, snap fit, pin, thread, bayonet fastener, clip, and/or locking key. With the dilator hub 370 fixed with respect to the sheath hub 20, the user can be confident that further advancement (or retraction) of the expansion element 365 will not occur during the heating step, including preparation and cool down.

In some implementations, the method further includes removing the dilator 350 from the lumen 212 of the sheath 208 after the heating step is complete. That is, in some implementations, the dilator 350 remains within the sheath 208 during the heating step. In other implementations, the dilator 350 is removed from the lumen 212 of the sheath 208 before the heating step.

In some implementations, the method further includes removing the restraining member 385 from the sheath 208. For example, with the heating step complete and prior to the medical procedure, the restraining member 385 can be removed from inner layer 202 and/or the strain relief layer 26. In some examples, the restraining member 385 is removed from the sheath 208 before the heating step. In some implementations, the restraining member 385 is removed before the dilator 350 is removed from the sheath 208. In some implementations, the restraining member 385 is removed after the dilator 350 is removed from the sheath 208.

In some implementations, the restraining member 385 includes a release feature 386 as described herein. Where the release feature 386 comprises a weakened portion (for example, perforation, score line, slit, etc.), the restraining member 385 can be removed from the sheath 208 by an outward force exerted on the restraining member 385 in a direction away from the sheath 208. The outward force causes the restraining member 385 to separate along the weakened portion. Where the release feature 386 comprises pull tab or pull line integral with the restraining member 385, the restraining member 385 is removed from the sheath 208 as the pull tab/pull line are activated and pulled away from the sheath 208. In some examples, the restraining member 385 is removed from the sheath 208 by cutting or tearing the restraining member 385. For example, where the restraining member 385 comprises a heat shrink tubing, the restraining member 385 can be removed from the sheath 208 by cutting the heat shrink tubing from the sheath 208.

In some implementations, as described herein, the restraining member 385 is incorporated into the packaging tray 400, such that removing the sheath 208 from the tray 400 at least partially removes the restraining member 385 from the sheath 208, including at least partially removing the restraining member 385 from the inner layer 202 and/or the strain relief layer 26.

With the restraining member 385 removed, the sheath 208 can be used to deliver a medical device to a procedure site within a patient's blood vessel. While the method of delivering a medical device using a pre-dilated sheath is described in reference to sheath 208 provided in FIGS. 15-23, it is contemplated that the method may also be used with sheath 8, provided in reference to FIGS. 11-14, or any other expandable sheath described herein.

The method of using the pre-dilated sheath 208 to deliver a medical device is described herein. The method includes at least partially inserting a pre-dilated sheath 208 into the blood vessel of the patient such that the distal end of the sheath 208 is positioned at a location proximate the treatment site. The sheath 208 can be pre-dilated using the dilator 350 and method described herein. Because the sheath 208 has been pre-dilated, a medical can be introduced into the central lumen 212 of the (pre-dilated) sheath 208 and the patient's blood vessel with greater ease and at lower push force.

The method further includes advancing a medical device through a portion of the sheath 208 corresponding to the strain relief layer 26 and exerting an outwardly directed radial force against the central lumen of the sheath 208 (for example, inner layer) and causing the sheath 208 (including the inner layer and/or the strain relief layer 26) proximate the medical device to locally expand from an unexpanded configuration (FIGS. 17-19 and 22) to an expanded configuration (FIGS. 20 and 23). In some examples, the medical device is contracted or compressed radially as it passes through the strain relief layer 20, from the proximal portion 242, through the tapered portion 248 and into the smaller diameter distal portion 246. As the medical device passes through the corresponding portion of the lumen of sheath 208, the sheath 208 and strain relief layer 26 locally contracts towards the unexpanded configuration.

The method further includes, advancing the medical device beyond the distal end 342 of the strain relief layer 26 and into the lumen of the body portion of the sheath 208 (beyond the strain relief layer 26) and ultimately beyond the distal opening in the sheath 208 to the treatment site. As the medical device is advanced through the sheath 208 beyond the strain relief layer 26, and through the distal opening, the sheath 208 locally expands from the unexpanded configuration (FIGS. 11-13A and FIGS. 17-19) to the expanded configuration (FIGS. 14, 20) at a location proximate the medical device in response to the outwardly directed radial force of the medical device exerted against the inner layer/central lumen of the sheath 208. Because the sheath 208 has been pre-dilated as described herein, unfolding and any undesirable separating along seam 234 or other opening between adjacent layers of the dilated sheath 208 is limited.

In some implementations, at least one of the inner layer and/or outer layer includes at least one folded portion. For example, in some implementations, the sheath includes ridges 126 and valleys 128 of the fourth (outer) layer 108 of the sheath 8 illustrated in FIGS. 11-14, and folded portion 218 of the inner layer 202 of the sheath 208 illustrated in FIGS. 15-23. Locally expanding the lumen of the sheath causes a length of the folded portion to at least partially unfold. Similarly, locally contracting the sheath at least partially back to the unexpanded configuration causes a length of the folded portion to urge back towards a folded configuration.

In some implementations, the outer layer 204 is a discontinuous outer layer and includes an overlapping portion (for example, overlapping portion 220) and an underlying portion (for example, underlying portion 222). When the sheath 208 is in the unexpanded configuration, the overlapping portion 220 overlaps the underlying portion 222 with the folded portion 218 of the inner layer disposed between the overlapping portion 220 and the underlying portion 222 (FIGS. 17, 19, 22, 23). As the sheath 208 locally expands to/toward the expanded configuration, a length of the overlapping portion 220 moves circumferentially with respect to the underlying portion 222 unfolding. As illustrated in FIG. 20, when the sheath 208 is fully expanded, the inner layer 202 extends into the gap 232 formed between the longitudinal edges of the overlapping portion 220 and the underlying portion 222 of the outer layer 204.

As the medical device passes through the lumen of the sheath 208, the sheath 208 locally contracts at least partially back to the unexpanded configuration (FIGS. 11-13A and FIGS. 17-19).

The method further includes advancing the medical device through the distal tip 9/distal opening of the sheath 208 and delivering the medical device to the treatment site. The position of the medical device can be moved or adjusted until the medical device is adequately positioned within the patient. With the medical device delivered to the treatment site, any delivery system/components coupled to the medical device are then removed from the medical device and withdrawn from the lumen of the sheath 208. The sheath 208 is removed from the patient and the opening in the blood vessel, and the skin is closed.

The medical device described herein can include a prosthetic device mounted in a radially crimped state on a delivery apparatus, and the act of advancing the prosthetic device through the lumen of the sheath 208 comprises advancing the delivery apparatus and the prosthetic device through lumen of the sheath 208 and into the vasculature of the patient. In some examples, the prosthetic device comprises a prosthetic heart valve and the method further comprises implanting the prosthetic heart valve at a treatment site within the patient. As described herein, the prosthetic heart valve is mounted on a balloon catheter of the delivery apparatus as the prosthetic heart valve is advanced through the sheath 208.

In view of the many possible implementations to which the principles of the disclosed disclosure can be applied, it should be recognized that the illustrated implementations are only preferred examples of the disclosure and should not be taken as limiting the scope of the disclosure. Rather, the scope of the disclosure is defined by the following claims. We, therefore, claim as our disclosure all that comes within the scope and spirit of these claims.

### Exemplary Aspects

In view of the many possible aspects to which the principles of the disclosed disclosure can be applied, it should be recognized that the illustrated aspects are only preferred examples of the disclosure and should not be taken as limiting the scope of the disclosure. Rather, the scope of the disclosure is defined by the following claims. We, therefore, claim as our disclosure all that comes within the scope and spirit of these claims.

Example 1. A method of manufacturing a pre-dilated expandable sheath for delivering a medical device including: providing a radially expandable sheath including: a continuous inner layer defining a lumen therethrough, the inner layer including a proximal portion and a main body portion, and having a folded portion extending along a length of the inner layer; a tubular strain relief layer provided over the proximal portion of the inner layer; wherein at least a portion of the sheath is configured to locally expand from an unexpanded configuration in which the lumen has a first diameter to an expanded configuration in which the lumen has a second, larger, diameter, and then locally contract at least partially back toward the unexpanded configuration; providing a restraining member positioned over a distal end of the strain relief layer, the restraining member limiting expansion of an adjacent portion including at least one of the inner layer and strain relief layer; introducing a dilator into a proximal end of the lumen of the sheath, the dilator including an expansion element provided thereon; advancing the dilator through the proximal portion of the inner layer such that the expansion element provided on the dilator exerts an outwardly directed radial force against the lumen and causes the inner layer proximate the expansion element to locally expand from an unexpanded configuration to an expanded configuration, wherein the restraining member limits expansion of the sheath proximate the restraining member; and heating the sheath.

Example 2. The method according to any example herein, particularly example 1, wherein the restraining member limits the unfolding of the folded portion of the inner layer proximate the restraining member when the sheath moves from the unexpanded configuration to the expanded configuration during advancement of the dilator through the proximal portion of the inner layer.

Example 3. The method according to any example herein, particularly examples 1-2, wherein the restraining member is provided over a length of sheath at a location corresponding to the distal end of the strain relief layer and extends along a length of the strain relief layer from the distal end toward a proximal end of the strain relief layer, and along a second length of the sheath from the distal end of the strain relief layer toward a distal end of the sheath.

Example 4. The method according to any example herein, particularly examples 1-3, wherein providing the restraining member over the sheath includes coupling the restraining member to at least one of the inner layer or the strain relief layer.

Example 5. The method according to any example herein, particularly example 4, wherein an inner surface of the restraining member includes an adhesive for coupling the restraining member to the sheath.

Example 6. The method according to any example herein, particularly example 4, wherein the restraining member includes a shrink tubing, wherein coupling the restraining member to at least one of the inner layer or the strain relief layer includes providing a shrink process to the restraining member.

Example 7. The method according to any example herein, particularly examples 1-6, further including: removing the restraining member from the inner layer and the strain relief layer.

Example 8. The method according to any example herein, particularly example 7, wherein the restraining member is removed after the heating step.

Example 9. The method according to any example herein, particularly example 7, wherein the restraining member is removed before the heating step.

Example 10. The method according to any example herein, particularly example 9, wherein a release feature is incorporated into a packaging sized and configured to receive the sheath, wherein providing a radially expandable sheath includes removing the sheath from the packaging, wherein removing the sheath from the packaging removes the restraining member from the inner layer and the strain relief layer.

Example 11. The method according to any example herein, particularly examples 1-10, wherein advancing the dilator through the proximal portion of the inner layer includes advancing the expansion element of the dilator toward the distal end of the strain relief layer, such that the distal end of the strain relief layer is expanded.

Example 12. The method according to any example herein, particularly examples 1-11, wherein advancing the dilator through the proximal portion of the inner layer includes advancing the expansion element of the dilator beyond the distal end of the strain relief layer, such that the distal end of the strain relief layer and a portion of the main body portion of the inner layer is expanded.

Example 13. The method according to any example herein, particularly example 12, wherein expanding the portion of the main body portion beyond the strain relief layer causes a corresponding length of the folded portion to at least partially unfold.

Example 14. The method according to any example herein, particularly examples 1-13, further including: removing the dilator from the lumen of the sheath after the heating step is complete.

Example 15. The method according to any example herein, particularly examples 1-14, further including: removing the dilator from the lumen of the sheath before the heating step.

Example 16. The method according to any example herein, particularly examples 1-15, wherein at least a portion of the strain relief layer is configured to locally expand from an unexpanded configuration at a first diameter to an expanded configuration at a second, larger, diameter, and then locally contract at least partially back to the unexpanded configuration.

Example 17. The method according to any example herein, particularly example 16, wherein at least a portion of the strain relief layer is configured to locally expand from the unexpanded configuration to the expanded configuration in response to the outwardly directed radial force exerted against the lumen by the dilator, and then locally contract at least partially back to the unexpanded configuration as the dilator moves within the lumen, wherein at least a portion of the sheath is configured to locally expand from the unexpanded configuration to the expanded configuration in response to an outwardly directed radial force exerted on the lumen of the inner layer by the dilator, and then locally contract at least partially back to the unexpanded configuration as the dilator moves within the lumen.

Example 18. The method according to any example herein, particularly examples 1-17, wherein the sheath further includes: an outer layer provided over the inner layer, where the outer layer is discontinuous and includes an overlapping portion and an underlying portion, and the overlapping portion overlaps the underlying portion, wherein at least a portion of the folded portion of the inner layer is positioned between the overlapping portion and the underlying portion, wherein the strain relief layer extends at least partially over the outer layer.

Example 19. The method according to any example herein, particularly example 18, wherein, in the unexpanded configuration, the folded portion extends circumferentially over an outer surface of the inner layer and/or outer layer, wherein, in the expanded configuration, local expansion causes a length of the folded portion to at least partially unfold, wherein, in the expanded configuration, local expansion of the sheath causes a length of the overlapping portion to move circumferentially with respect to the underlying portion.

Example 20. The method according to any example herein, particularly example 19, wherein, in the expanded configuration, local expansion of the sheath forms a gap between longitudinally extending edges of the outer layer, wherein at least a portion of the unfolded portion extends into the gap, wherein the restraining member limits expansion of the sheath and a width of the gap proximate the restraining member.

Example 21. The method according to any example herein, particularly examples 1-20, wherein the sheath further includes an elastic outer cover extending at least partially over the sheath where the outer cover locally expands and contracts as the medical device is advanced through the lumen, wherein the elastic outer cover exerts a radially inward force on the sheath.

Example 22. The method according to any example herein, particularly examples 1-20, wherein the sheath further includes a sheath hub fixedly coupled to the proximal end of the sheath, the sheath hub including a central lumen extending therethrough and coaxial with the lumen of the sheath, wherein the dilator shaft is sized and configured to be received within the central lumen of the sheath hub, wherein the dilator includes a dilator hub coupled to a proximal end of the dilator shaft, wherein the method further includes: advancing the dilator through the proximal portion until the dilator hub abuts the sheath hub; and coupling the dilator hub to the sheath hub before heating the sheath.

Example 23. The method according to any example herein, particularly examples 1-21, wherein heating the sheath includes heating the sheath at a temperature and for a duration corresponding to a sterilization process, wherein, during the heating, the sheath is not heated at a temperature or for a duration sufficient to bond layers of the folded portion.

Example 24. The method according to any example herein, particularly examples 1-23, wherein heating the sheath includes heating the sheath at a temperature of 60C.

Example 25. The method according to any example herein, particularly examples 1-24, wherein heating the sheath includes heating the sheath for a duration greater than 12 hours.

Example 26. A sheath system comprising: a radially expandable sheath including: a continuous inner layer defining a lumen therethrough, the inner layer including a proximal portion and a main body portion and a folded portion extending along a length of the inner layer; a tubular strain relief layer provided over the proximal portion of the inner layer; and a restraining member positioned over a distal end of the strain relief layer, the restraining member limiting expansion of an adjacent portion of at least one of the inner layer and strain relief layer; and a dilator sized and configured to be received within the lumen of the inner layer, the dilator including an elongated dilator shaft and an expansion element provided thereon; wherein at least a portion of the sheath is configured to locally expand from an unexpanded configuration in which the lumen has a first diameter to an expanded configuration in which the lumen has a second, larger, diameter in response to an outwardly directed radial force exerted on the lumen by the expansion element of the dilator, and then locally contract at least partially back toward unexpanded configuration as the dilator passes through the lumen, wherein the restraining member limits expansion of the sheath proximate the restraining member.

Example 27. The sheath system according to any example herein, particularly example 26, wherein the restraining member limits the unfolding of the folded portion of the inner layer proximate the restraining member when the sheath moves from the unexpanded to the expanded configuration.

Example 28. The sheath system according to any example herein, particularly examples 26-27, wherein the restraining member is provided over a length of sheath at a location corresponding to the distal end of the strain relief layer and extends along a length of the strain relief layer from the distal end toward a proximal end of the strain relief layer, and along a second length of the sheath from the distal end of the strain relief layer toward a distal end of the sheath.

Example 29. The sheath system according to any example herein, particularly examples 26-28, wherein the restraining member comprises at least one of a tape, a shrink tube, an elastic tube, or a packaging feature.

Example 30. The sheath system according to any example herein, particularly examples 26-29, wherein the restraining member is coupled to the sheath.

Example 31. The sheath system according to any example herein, particularly example 30, wherein an inner surface of the restraining member includes an adhesive for coupling the restraining member to the sheath.

Example 32. The sheath system according to any example herein, particularly example 30, wherein the restraining member includes a shrink tubing, wherein the restraining member is coupled to at least one of the inner layer or the strain relief layer by a shrink process.

Example 33. The sheath system according to any example herein, particularly examples 26-32, wherein the restraining member includes a release feature for removing the restraining member from the sheath.

Example 34. The sheath system according to any example herein, particularly example 33, wherein the release feature includes at least one of a weakened portion or a pull tab and/or line integral with the restraining member.

Example 35. The sheath system according to any example herein, particularly examples 33-34, wherein the release feature is incorporated into a packaging sized and configured to receive the sheath, wherein removing the sheath from the packaging removes the restraining member from the inner layer and the strain relief layer.

Example 36. The sheath system according to any example herein, particularly examples 26-35, wherein the dilator shaft includes a body portion adjacent a proximal end of the dilator shaft, and a tapered portion extending from a distal end of the dilator shaft toward the body portion, where the expansion element is provided on the body portion.

Example 37. The sheath system according to any example herein, particularly examples 26-36, wherein the expansion element is defined by the body portion of the dilator shaft.

Example 38. The sheath system according to any example herein, particularly examples 26-37, wherein the expansion element includes a projection extending from an outer surface of the dilator shaft.

Example 39. The sheath system according to any example herein, particularly examples 26-38, wherein the diameter of the expansion element is 22F.

Example 40. The sheath system according to any example herein, particularly examples 26-39, wherein at least a portion of the strain relief layer is configured to locally expand from an unexpanded configuration at a first diameter to an expanded configuration at a second, larger, diameter, and then locally contract at least partially back to the unexpanded configuration.

Example 41. The sheath system according to any example herein, particularly examples 40, wherein at least a portion of the strain relief layer is configured to locally expand from the unexpanded configuration to the expanded configuration in response to an outwardly directed radial force exerted against the lumen by the dilator, and then locally contract at least partially back to the unexpanded configuration as the dilator 350 moves within the lumen.

Example 42. The sheath system according to any example herein, particularly examples 26-41, wherein the strain relief layer includes: a proximal portion adjacent a proximal end of the strain relief layer; a distal portion adjacent a distal end of the strain relief layer; and a tapered portion extending between the distal portion and the proximal portion, wherein a diameter of the proximal portion is greater than a diameter of the distal portion.

Example 43. The sheath system according to any example herein, particularly examples 26-42, wherein the strain relief layer comprises a stiffer and/or less elastomeric material than the inner layer and restricts expansion of the inner layer.

Example 44. The sheath system according to any example herein, particularly examples 26-43, wherein the strain relief layer comprises a material having a higher durometer than the inner layer such that the strain relief layer restricts expansion of the sheath.

Example 45. The sheath system according to any example herein, particularly examples 26-44, wherein the strain relief layer comprises polyurethane.

Example 46. The sheath system according to any example herein, particularly examples 26-45, wherein as the strain relief layer moves from the unexpanded configuration to the expanded configuration, a length of the strain relief layer remains constant.

Example 47. The sheath system according to any example herein, particularly examples 26-46, wherein the sheath further includes: an outer layer provided over the inner layer; wherein the strain relief layer comprises a stiffer and/or less elastomeric material than the inner layer and outer layer and restricts expansion of at least one of the inner or outer layers, wherein the strain relief layer comprises a material having a higher durometer than the inner layer and/or the outer layer such that the strain relief layer restricts expansion of at least one of the inner or outer layers.

Example 48. The sheath system according to any example herein, particularly examples 26-46, wherein the sheath further includes: an outer layer provided over the inner layer, where the outer layer is discontinuous and includes an overlapping portion and an underlying portion, and the overlapping portion overlaps the underlying portion, wherein at least a portion of the folded portion of the inner layer is positioned between the overlapping portion and the underlying portion, wherein the strain relief layer extends at least partially over the outer layer.

Example 49. The sheath system according to any example herein, particularly example 48, wherein, in the unexpanded configuration, the folded portion extends circumferentially over an outer surface of the inner layer and/or outer layer.

Example 50. The sheath system according to any example herein, particularly examples 48-49, wherein, in the expanded configuration, local expansion causes a length of the folded portion to at least partially unfold forming an unfolded portion of the inner layer, wherein, in the expanded configuration, local expansion of the sheath causes a length of the overlapping portion to move circumferentially with respect to the underlying portion.

Example 51. The sheath system according to any example herein, particularly example 50, wherein, in the expanded configuration, local expansion of the sheath forms a gap between longitudinally extending edges of the outer layer, wherein at least a portion of the unfolded portion extends into the gap, wherein the restraining member limits expansion of the sheath and a width of the gap proximate the restraining member.

Example 52. The sheath system according to any example herein, particularly examples 26-51, wherein an overall length of the strain relief layer and/or sheath does not change when the sheath and/or strain relief layer moves between the unexpanded configuration and expanded configuration.

Example 53. The sheath system according to any example herein, particularly examples 26-52, wherein the lumen of the inner layer is cylindrical in the unexpanded and expanded configurations.

Example 54. The sheath system according to any example herein, particularly examples 26-53, wherein the inner layer comprises PTFE and the outer layer comprises HDPE and/or Tecoflex.

Example 55. The sheath system according to any example herein, particularly examples 47-57, wherein the inner and outer layers are bonded together.

Example 56. The sheath system according to any example herein, particularly examples 47-55, wherein the inner and outer layers are thermally bonded together.

Example 57. The sheath system according to any example herein, particularly examples 47-56, wherein the inner and outer layers are bonded together by an adhesive.

Example 58. The sheath system according to any example herein, particularly examples 47-57, wherein the strain relief layer is bonded to the outer layer and/or inner layer.

Example 59. The sheath system according to any example herein, particularly examples 47-58, wherein the strain relief layer is thermally and/or adhesively bonded to the outer layer and/or inner layer.

Example 60. The sheath system according to any example herein, particularly examples 47-59, wherein the inner layer comprises a woven fabric and/or braided filaments.

Example 61. The sheath system according to any example herein, particularly examples 47-60, wherein the inner layer comprises yarn filaments of PTFE, PET, PEEK, and/or nylon.

Example 62. The sheath system according to any example herein, particularly examples 47-61, wherein the outer layer comprises polyurethane.

Example 63. The sheath system according to any example herein, particularly examples 26-62, the sheath system further includes an elastic outer cover extending at least partially over the sheath where the outer cover locally expands and contracts as the dilator is advanced through the lumen, wherein the elastic outer cover exerts a radially inward force on the sheath, wherein the elastic outer cover comprises PEBAX, polyurethane, silicone, or polyisoprene, or combination thereof.

Example 64. The sheath system according to any example herein, particularly examples 26-63, wherein the sheath further includes a sheath hub fixedly coupled to the proximal end of the sheath, the sheath hub including a central lumen extending therethrough and coaxial with the lumen of the sheath, wherein the dilator shaft is sized and configured to be received within the central lumen of the sheath hub, wherein the dilator includes a dilator hub coupled to a proximal end of the dilator shaft, wherein the dilator hub is configured to be coupled to the sheath hub.

Example 65. The sheath system according to any example herein, particularly examples 64, wherein the sheath hub includes one or more seals for forming a seal around an outer surface of a delivery apparatus movable through the central lumen of the sheath hub.

Example 66. A sheath system kit comprising: a radially expandable sheath including: a continuous inner layer defining a lumen therethrough, the inner layer including a proximal portion and a main body portion and a folded portion extending along a length of the inner layer; a tubular strain relief layer provided over the proximal portion of the inner layer; a restraining member positioned over a distal end of the strain relief layer, the restraining member limiting expansion of an adjacent portion of at least one of the inner layer and strain relief layer; a dilator sized and configured to be received within the lumen of the inner layer, the dilator including an elongated shaft and an expansion element provided thereon; and a tray sized and configured to receive the sheath and the dilator, the tray including a release mechanism coupled to the restraining member, wherein upon removal of the sheath from the tray, the release mechanism retains the restraining member thereby removing it from the sheath; wherein at least a portion of the sheath is configured to locally expand from an unexpanded configuration in which the lumen has a first diameter to an expanded configuration in which the lumen has a second, larger, diameter in response to an outwardly directed radial force exerted on the lumen by the expansion element of the dilator, and then locally contract at least partially back toward unexpanded configuration as the dilator passes through the lumen, wherein the restraining member limits expansion of the sheath proximate the restraining member.

Example 67. A method of delivering a medical device through a sheath, the method comprising: providing a radially expandable sheath including: a continuous inner layer defining a lumen therethrough, the inner layer including a proximal portion and a main body portion and a folded portion extending along a length of the inner layer; a tubular strain relief layer provided over the proximal portion of the inner layer; and a restraining member positioned over a distal end of the strain relief layer, the restraining member limiting expansion of an adjacent portion of at least one of the inner layer and strain relief layer; removing a dilator received from the lumen of the inner layer, where the restraining member limits expansion of the sheath due to an outwardly directed radial force exerted by the dilator; removing the restraining member from the sheath; introducing a medical device into a proximal end of a central lumen of the sheath; advancing the medical device through the proximal portion of the inner layer causing the inner layer and the strain relief layer proximate the medical device to locally expand from an unexpanded configuration to an expanded configuration, and locally contracting the strain relief layer towards the unexpanded configuration as the medical device passes through the corresponding portion of the lumen of sheath; advancing the medical device beyond a distal end of the strain relief layer; advancing a medical device through the main body portion of the lumen of the sheath causing the main body portion of the sheath to locally expand from the unexpanded configuration to the expanded configuration at a location proximate the medical device in response to the outwardly directed radial force of the medical device exerted against the inner layer locally contracting the sheath at least partially back to the unexpanded configuration as the medical device passes through the lumen; and advancing the medical device beyond a distal opening in the sheath.

Example 68. The method according to any example herein, particularly example 67, wherein at least a portion of the sheath is configured to locally expand from an unexpanded configuration in which the lumen has a first diameter to an expanded configuration in which the lumen has a second, larger, diameter in response to an outwardly directed radial force exerted on the lumen by the expansion element of the dilator, and then locally contract at least partially back toward unexpanded configuration as the dilator passes through the lumen.

Example 69. A method of inserting a medical device into a blood vessel of a patient, the method includes: providing a radially expandable sheath including: a continuous inner layer defining a lumen therethrough, the inner layer including a proximal portion and a main body portion and a folded portion extending along a length of the inner layer; a tubular strain relief layer provided over the proximal portion of the inner layer; and a restraining member positioned over a distal end of the strain relief layer, the restraining member limiting expansion of an adjacent portion of at least one of the inner layer and strain relief layer; removing a dilator received from the lumen of the inner layer, where the restraining member limits expansion of the sheath due to an outwardly directed radial force exerted by the dilator; removing the restraining member from the sheath; inserting the sheath at least partially into the blood vessel of the patient; introducing a medical device into a proximal end of the central lumen of the sheath; advancing the medical device through the proximal portion of the inner layer and thereby exerting an outwardly directed radial force by the medical device against the central lumen causing the inner layer and the strain relief layer proximate the medical device to locally expand from an unexpanded configuration to an expanded configuration, and locally contracting the strain relief layer towards the unexpanded configuration as the medical device passes through the corresponding portion of the lumen of sheath; advancing the medical device beyond the distal end of the strain relief layer; advancing a medical device through the main body portion lumen of the sheath causing the main body portion of the sheath to locally expand from an unexpanded configuration to an expanded configuration at a location proximate the medical device in response to the outwardly directed radial force of the medical device exerted against the inner layer, and locally contracting the sheath at least partially back to the unexpanded configuration as the medical device passes through the lumen; and advancing the medical device beyond a distal opening in the sheath to a treatment site within the blood vessel.

Example 70. The method according to any example herein, particularly example 69, wherein the dilator expands the distal end of the strain relief layer.

Example 71. The method according to any example herein, particularly examples 69-70, wherein the inner layer includes at least one folded portion, wherein locally expanding the lumen of the sheath causes a length of the folded portion to at least partially unfold.

Example 72. The method according to any example herein, particularly examples 69-71 wherein the sheath further includes: an outer layer provided over the inner layer, where the outer layer is discontinuous and includes an overlapping portion and an underlying portion, wherein when the sheath is in the unexpanded configuration, the overlapping portion overlaps the underlying portion with the folded portion of the inner layer disposed between the overlapping portion and the underlying portion, wherein the strain relief layer extends at least partially over the outer layer, and wherein the medical device is a prosthetic device mounted in a radially crimped state on a delivery apparatus.

Example 73. The method according to any example herein, particularly example 72, wherein advancing the prosthetic device through the lumen of the sheath comprises advancing the delivery apparatus and the prosthetic device through the lumen of the sheath and into a vasculature of the patient.

Example 74. The method according to any example herein, particularly example 73, wherein the prosthetic device comprises a prosthetic heart valve and the method further comprises implanting the prosthetic heart valve at the treatment site within the patient.

Example 75. The method according to any example herein, particularly examples 73-74, wherein the prosthetic heart valve is mounted on a balloon catheter of the delivery apparatus as the prosthetic heart valve is advanced through the sheath.

Example 76. The method according to any example herein, particularly examples 69-75, wherein the sheath is inserted into a femoral artery of the patient.

What is claimed is:

1. A method of manufacturing a pre-dilated expandable sheath for delivering a medical device including:
providing a radially expandable sheath including:
a continuous inner layer defining a lumen therethrough, the inner layer including a proximal portion and a main body portion, and having a folded portion extending along a length of the inner layer;
a tubular strain relief layer provided over the proximal portion of the inner layer;
wherein at least a portion of the sheath is configured to locally expand from an unexpanded configuration in which the lumen has a first diameter to an expanded configuration in which the lumen has a second, larger, diameter, and then locally contract at least partially back toward the unexpanded configuration;
providing a restraining member positioned over a distal end of the strain relief layer, the restraining member limiting expansion of an adjacent portion including at least one of the inner layer and strain relief layer;
introducing a dilator into a proximal end of the lumen of the sheath, the dilator including an expansion element provided thereon;
advancing the dilator through the proximal portion of the inner layer such that the expansion element provided on the dilator exerts an outwardly directed radial force against the lumen and causes the inner layer proximate the expansion element to locally expand from an unexpanded configuration to an expanded configuration, wherein the restraining member limits expansion of the sheath proximate the restraining member; and
heating the sheath.

2. The method of claim 1, wherein the restraining member limits the unfolding of the folded portion of the inner layer proximate the restraining member when the sheath moves from the unexpanded configuration to the expanded configuration during advancement of the dilator through the proximal portion of the inner layer.

3. The method of claim 1, wherein the restraining member is provided over a length of sheath at a location corresponding to the distal end of the strain relief layer and extends along a length of the strain relief layer from the distal end toward a proximal end of the strain relief layer, and along a second length of the sheath from the distal end of the strain relief layer toward a distal end of the sheath.

4. The method of claim 1, wherein providing the restraining member over the sheath includes coupling the restraining member to at least one of the inner layer or the strain relief layer.

5. The method of claim 4, wherein the restraining member includes at least one of an adhesive or a shrink tubing,
wherein, when the restraining member includes an adhesive, an inner surface of the restraining member includes the adhesive for coupling the restraining member to the sheath,
wherein, when the restraining member includes the shrink tubing, coupling the restraining member to at least one of the inner layer or the strain relief layer includes providing a shrink process to the restraining member.

6. The method of claim 1, further including:
removing the restraining member from the inner layer and the strain relief layer,
wherein a release feature is incorporated into a packaging sized and configured to receive the sheath,
wherein providing a radially expandable sheath includes removing the sheath from the packaging, wherein removing the sheath from the packaging removes the restraining member from the inner layer and the strain relief layer.

7. The method of claim 1, wherein advancing the dilator through the proximal portion of the inner layer includes advancing the expansion element of the dilator toward the distal end of the strain relief layer, such that the distal end of the strain relief layer is expanded.

8. The method of claim 1, wherein advancing the dilator through the proximal portion of the inner layer includes advancing the expansion element of the dilator beyond the distal end of the strain relief layer, such that the distal end of the strain relief layer and a portion of the main body portion of the inner layer is expanded,
wherein expanding the portion of the main body portion beyond the strain relief layer causes a corresponding length of the folded portion to at least partially unfold.

9. The method of claim 1, wherein the sheath further includes:
an outer layer provided over the inner layer, where the outer layer is discontinuous and includes an overlapping portion and an underlying portion, and the overlapping portion overlaps the underlying portion,
wherein at least a portion of the folded portion of the inner layer is positioned between the overlapping portion and the underlying portion,
wherein the strain relief layer extends at least partially over the outer layer,
wherein, in the unexpanded configuration, the folded portion extends circumferentially over an outer surface of the inner layer and/or outer layer,
wherein, in the expanded configuration, local expansion causes a length of the folded portion to at least partially unfold,
wherein, in the expanded configuration, local expansion of the sheath causes a length of the overlapping portion to move circumferentially with respect to the underlying portion and forms a gap between longitudinally extending edges of the outer layer,
wherein at least a portion of the unfolded portion extends into the gap,
wherein the restraining member limits expansion of the sheath and a width of the gap proximate the restraining member.

10. The method of claim 1, wherein heating the sheath includes heating the sheath at a temperature and for a duration corresponding to a sterilization process,
wherein, during the heating, the sheath is not heated at a temperature or for a duration sufficient to bond layers of the folded portion.

11. A sheath system comprising:
a radially expandable sheath including:
a continuous inner layer defining a lumen therethrough, the inner layer including a proximal portion and a main body portion and a folded portion extending along a length of the inner layer;
a tubular strain relief layer provided over the proximal portion of the inner layer; and
a restraining member positioned over a distal end of the strain relief layer, the restraining member limiting expansion of an adjacent portion of at least one of the inner layer and strain relief layer; and
a dilator sized and configured to be received within the lumen of the inner layer, the dilator including an elongated dilator shaft and an expansion element provided thereon;
wherein at least a portion of the sheath is configured to locally expand from an unexpanded configuration in which the lumen has a first diameter to an expanded configuration in which the lumen has a second, larger, diameter in response to an outwardly directed radial force exerted on the lumen by the expansion element of the dilator, and then locally contract at least partially back toward unexpanded configuration as the dilator passes through the lumen,
wherein the restraining member limits expansion of the sheath proximate the restraining member.

12. The system of claim 11, wherein the restraining member is coupled to the sheath and limits the unfolding of the folded portion of the inner layer proximate the restraining member when the sheath moves from the unexpanded to the expanded configuration.

13. The system of claim 11, wherein the restraining member includes a release feature including at least one of a weakened portion or a pull tab and/or line integral with the restraining member for removing the restraining member from the sheath,
wherein the release feature is incorporated into a packaging sized and configured to receive the sheath, and
wherein removing the sheath from the packaging removes the restraining member from the inner layer and the strain relief layer.

14. The system of claim 11, wherein the dilator shaft includes a body portion adjacent a proximal end of the dilator shaft, and a tapered portion extending from a distal end of the dilator shaft toward the body portion, where the expansion element is provided on the body portion and includes a projection extending from an outer surface of the dilator shaft.

15. The system of claim 11, wherein at least a portion of the strain relief layer is configured to locally expand from an unexpanded configuration at a first diameter to an expanded configuration at a second, larger, diameter, and then locally contract at least partially back to the unexpanded configuration,
wherein at least a portion of the strain relief layer is configured to locally expand from the unexpanded configuration to the expanded configuration in response to an outwardly directed radial force exerted against the lumen by the dilator, and then locally contract at least partially back to the unexpanded configuration as the dilator moves within the lumen.

16. The system of claim 11, wherein the sheath further includes:
an outer layer provided over and bonded to the inner layer;
wherein the strain relief layer comprises a stiffer and/or less elastomeric material than the inner layer and outer layer and restricts expansion of at least one of the inner or outer layers,
wherein the strain relief layer comprises a material having a higher durometer than the inner layer and/or the outer layer such that the strain relief layer restricts expansion of at least one of the inner or outer layers.

17. The system of claim 11, wherein the sheath further includes:
an outer layer provided over the inner layer, where the outer layer is discontinuous and includes an overlapping portion and an underlying portion, and the overlapping portion overlaps the underlying portion,
wherein at least a portion of the folded portion of the inner layer is positioned between the overlapping portion and the underlying portion,
wherein the strain relief layer extends at least partially over the outer layer.

18. The system of claim 17, wherein, in the expanded configuration, local expansion causes a length of the folded portion to at least partially unfold forming an unfolded portion of the inner layer,
wherein, in the expanded configuration, local expansion of the sheath causes a length of the overlapping portion to move circumferentially with respect to the underlying portion and forms a gap between longitudinally extending edges of the outer layer, wherein at least a portion of the unfolded portion extends into the gap,
wherein the restraining member limits expansion of the sheath and a width of the gap proximate the restraining member.

19. The system of claim 11, wherein an overall length of the strain relief layer and/or sheath does not change when the sheath and/or strain relief layer moves between the unexpanded configuration and expanded configuration.

20. A sheath system kit comprising:
a radially expandable sheath including:
a continuous inner layer defining a lumen therethrough, the inner layer including a proximal portion and a main body portion and a folded portion extending along a length of the inner layer;
a tubular strain relief layer provided over the proximal portion of the inner layer;
a restraining member positioned over a distal end of the strain relief layer, the restraining member limiting expansion of an adjacent portion of at least one of the inner layer and strain relief layer;
a dilator sized and configured to be received within the lumen of the inner layer, the dilator including an elongated shaft and an expansion element provided thereon; and
a tray sized and configured to receive the sheath and the dilator, the tray including a release mechanism coupled to the restraining member, wherein upon removal of the sheath from the tray, the release mechanism retains the restraining member thereby removing it from the sheath;
wherein at least a portion of the sheath is configured to locally expand from an unexpanded configuration in which the lumen has a first diameter to an expanded configuration in which the lumen has a second, larger, diameter in response to an outwardly directed radial force exerted on the lumen by the expansion element of the dilator, and then locally contract at least partially back toward unexpanded configuration as the dilator passes through the lumen,
wherein the restraining member limits expansion of the sheath proximate the restraining member.
